(19) United States
(12) Patent Application Publication
(10) Pub. No.: US 2025/0296995 A1
(43) Pub. Date: Sep. 25, 2025

(54) ANTIGEN BINDING PROTEIN AND USES THEREOF

(71) Applicant: GLAXOSMITHKLINE INTELLECTUAL PROPERTY DEVELOPMENT LIMITED, Stevenage (GB)

(72) Inventors: Joanna CORDY, Stevenage (GB); Maria De Gracia GOMEZ LORENZO, Madrid (ES); Gerald GOUGH, Stevenage (GB)

(73) Assignee: GLAXOSMITHKLINE INTELLECTUAL PROPERTY DEVELOPMENT LIMITED, Stevenage (GB)

(21) Appl. No.: 19/105,056

(22) PCT Filed: Aug. 23, 2023
(86) PCT No.: PCT/EP2023/073096

§ 371 Date: Feb. 20, 2025

(30) Foreign Application Priority Data
Aug. 25, 2022 (EP) 22382798.1

(51) Int. Cl. C07K 16/20; A61K 39/00; A61P 37/04

(52) U.S. Cl. CPC C07K 16/205; A61P 37/04; A61K 2039/505; C07K 2317/52; C07K 2317/92

(57) ABSTRACT

The present invention provides antigen binding proteins such as antibodies targeting Plasmodium sporozoites, in particular Plasmodium circumsporozoite protein. The invention also provides nucleic acids that encode such antibodies. In addition, the invention provides antibodies according to the invention for use in the prophylaxis of malaria.

### FIELD OF THE INVENTION

The present invention relates to the field of malaria medication, in particular to antibodies binding to Plasmodium falciparum sporozoites, in particular to Plasmodium falciparum circumsporozoite protein for the prevention of malaria.

### BACKGROUND TO THE INVENTION

Malaria is one of the most severe public health problems worldwide. Malaria is caused by parasitic protozoans of the genus Plasmodium. The genus Plasmodium includes about 200 species with P. falciparum, P. vivax, P. ovale and P. malariae together accounting for nearly all human infections with Plasmodium species. Among those Plasmodium species, P. falciparum accounts for the overwhelming majority of malaria deaths, particularly in children under five years of age. Malaria symptoms typically include fever, feeling tired, vomiting, and headaches. In severe cases it can cause yellow skin, seizures, coma, or death.

Malaria is a mosquito-borne disease transmitted by the bit of an infected female Anopheles mosquito. In the case of human malaria by P. falciparum infection, the female Anopheles mosquito injects a small number of sporozoites (10-100) into the skin. Some of those parasites travel to the liver to invade hepatocytes (Crompton et al. (2014) Annu Rev Immunol 32, 157-187). In the hepatocytes, the sporozoites forms a parasitophorous vacuole where they develop, multiply asexually (tissue schizogony) and mature into schizonts which, when matured, rupture to release thousands of merozoites that ultimately are released into the blood stream. Merozoites infect red blood cells, maturing from rings to trophozoites and finally into schizonts, which rupture releasing merozoites that will infect new red blood cells in cycles of 48 hours, perpetuating the cycle. Other merozoites develop into sexual erythrocytic stages (gametocytes). When a mosquito bites an infected human, gametocytes are taken up with the blood and mature in the mosquito gut. The male and female gametocytes fuse and form an ookinete—a fertilized, motile zygote. Ookinetes develop further into oocysts and finally into new sporozoites that migrate to the insect's salivary glands to infect a new vertebrate host.

Malaria symptoms are caused by blood stage parasites. In contrast, sporozoites are not associated with clinical symptoms, however, in sporozoite and liver stages of the life cycle of Plasmodium parasite numbers in the host are low and their eradication can completely abrogate infection. Accordingly, the sporozoite and liver stages of the P. falciparum parasite represent key targets of current malaria prophylactic candidates, as an intervention that successfully protects against these stages would be able to prevent both malaria infection and transmission. Therefore, vaccines or molecules that block sporozoites are at the center of the development of malaria prophylactic efforts.

The Plasmodium circumsporozoite protein (CSP) is a membrane bound protein only present during the sporozoite stage of Plasmodium. CSP forms a dense coat on the surface of the parasite and has been hypothesized to mediate many of the initial interactions between the sporozoite and its two hosts (Menard, 2000, Microbes Infect. 2:633-642; Sinnis and Nardin, 2002, Chem Immunol 80:70-96)). The structure and function of CSP is highly conserved across the various strains of Plasmodium that infect humans, non-human primates and rodents. The amino-acid sequence of CSP comprises an immunodominant central repeat region, that is diverse across Plasmodium species (NANP-repeat region in case of P. falciparum). Flanking the repeats are two conserved motifs and a known cell-adhesive motif C-terminal to the repeats termed the type I thrombospondin repeat (TSR). Those conserved motifs are implicated in protein processing as the parasite travels from the mosquito to the mammalian vector. CSP is known to play a crucial role in the migration of the sporozoites from the midgut walls of infected mosquitoes to the mosquito salivary glands. Additionally, CSP is involved in hepatocyte binding in the mammalian host with the N-terminus CSP initially facilitating parasite binding. On the hepatocyte surface proteolytic cleavage at region I of the N-terminus exposes the adhesive C-terminal domain thereby priming the parasites for invasion of the liver (Coppi et al, 2005, J Exp Med 201, 27-33).

At present, the most advanced malaria vaccine candidate is RTS,S (RTS,S/AS01; trade name Mosquirix), which is a recombinant protein-based malaria vaccine. RTS,S is a hybrid protein particle, formulated in a multi-component adjuvant named AS01. The RTS,S vaccine antigen consists of 19 NANP amino acid repeat units followed by the complete C-terminal domain minus the GPI anchor of the CSP antigen, fused to the Hepatitis B virus S protein. Multisite clinical trials in sub-Saharan Africa have shown that RTS,S confers modest and short-lived protection against clinical malaria (RTS,S Clinical Trials Partnership, 2015, Lancet. 386(9988):31-45).

Another factor that has complicated the development of malaria treatments and prophylactics is the difficulty in identifying the nature of the mechanisms that confer robust immune protection although progress has recently been made in identifying immune correlates related to antibody specificity and function (Suscovich et al, 2020, Sci Transl Med 12(553):eabb4757).

Recently, anti-malaria antibodies were described, which are specific for P. falciparum CSP (Tan et al, 2018, Nat Med 24(4):401-407; Kisalu et al, 2018, Nat Med 24(4):408-416; Wang et al, 2020, Immunity 13; 53(4):733-744; Pholcharee et al, 2021, Nat Commun 16; 12(1):1063). However, these CSP binding antibodies although described as protective in mouse pre-clinical models of infection all have a proposed mechanism of action where the sporozoite is immobilised in the skin or prevented from binding to and/or infecting target cells in the liver by mechanisms mediated exclusively by the antigen binding Fab region of the antibody (e.g. CIS43, L9). There is increasing recognition that antibody Fc-mediated effector mechanisms can have an important role to play in protection from infection and moreover that manipulation of those mechanisms using amino acid substitutions in the Fc region of the antibody can significantly improve the overall level of protection conferred by an antibody. The immune correlates studies recently published (Suscovich et al, 2020, Sci Transl Med 12(553):eabb4757) point to such a role in malaria whilst other studies have previously demonstrated this principle in other infectious disease settings (Hiatt et al, 2014, PNAS 111; 5992-5997; DiLillo et al, 2014, Nat Med 20(2) 143-151).

In view of the above, it is the object of the present invention to overcome the limitations of the prior art treatments for malaria outlined above by providing not only antibodies with high affinity but those which confer the benefits of other protective mechanisms and improved safety as well as ease of development given the inherent issues with administration in malaria endemic countries.

### SUMMARY OF THE INVENTION

In one aspect of the invention there is provided a Plasmodium falciparum antigen binding protein.

In one aspect there is provided a Plasmodium falciparum circumsporozoite protein (CSP) antigen binding protein comprising:
a Heavy Chain (HC) sequence of SEQ ID NO: 9; and
a Light Chain (LC) sequence of SEQ ID NO: 10.

In one aspect there is provided a nucleic acid sequence encoding a heavy chain of SEQ ID NO:9; and/or a nucleic acid sequence encoding a light chain of SEQ ID NO: 10.

In one aspect there is provided an expression vector or vectors comprising a nucleic acid sequence encoding the heavy chain of SEQ ID NO:9; and/or a nucleic acid sequence encoding the light chain of SEQ ID NO: 10.

In one aspect there is provided a recombinant host cell comprising the nucleic acid sequence(s) encoding the heavy chain of SEQ ID NO:9; and/or a nucleic acid sequence encoding the light chain of SEQ ID NO: 10 or an expression vector or vectors comprising a nucleic acid sequence encoding the heavy chain of SEQ ID NO:9; and/or a nucleic acid sequence encoding the light chain of SEQ ID NO: 10.

In one aspect there is provided a method for the production of a circumsporozoite protein (CSP) antigen binding protein according to the invention as disclosed herein, which method comprises culturing a host cell under conditions suitable for expression of said nucleic acid sequence(s) or vector(s) according to the invention as herein described, whereby a polypeptide comprising the CSP antigen binding protein is produced.

In one aspect there is provided a pharmaceutical composition comprising the CSP antigen binding protein according to the invention as herein described and a pharmaceutically acceptable excipient.

In one aspect there is provided a method for the prevention of malaria in a subject in need thereof comprising administering to said subject a therapeutically effective amount of the CSP antigen binding protein according to the invention as herein described, or a pharmaceutical composition comprising the CSP antigen binding protein according to the invention as herein described.

In one aspect the CSP antigen binding protein according to the invention as herein described or a pharmaceutical composition comprising the CSP antigen binding protein according to the invention as herein described is for use in the prophylaxis of malaria.

### DESCRIPTION OF FIGURES

FIG. 1: Diversity analysis of the hit panel of antibodies

FIG. 2: Heparin binding ELISA data on the hit panel of antibodies showing a range of molecules with higher heparin binding profile than the negative control including AB-000325, AB-000337, AB001446 and AB-001516 which were subsequently removed from further analysis. Antibodies were incubated on heparin-coated poly-L-lysine plates for two hours prior to detection with anti-F(ab)′2-HRP.

FIG. 3: Heparin binding ELISA data on the Fc variants panel of antibodies. Heparin ELISA binding data on a panel of mAb A antibodies carrying different amino acid substitutions to confer half-life extension and improved effector function

FIG. 4: Human serum stability on the Fc variant panel of antibodies. mAb-A Fc variants do not show big differences in stability in human plasma but different molecules have inherently different stabilities i.e. mAb-A vs mAb-B. In vitro human serum stability over 8 weeks time period using CSP coated plates for capture and sulfo-tagged anti human IgG1 Fc mAb for antibody detection is shown.

FIG. 5: In vivo evaluation of the impact of Fc enhancement on antibody efficacy in the P. berghei model FIG. 5a shows Liver burden measured by bio-luminescence at 44 hours. FIG. 5b shows day 5 post sporozoite challenge (percentage of RBC's infected) at 25 μg dose. FIG. 5c shows day 8 post sporozoite challenge (percentage of RBC's infected) at 100 μg dose. FIG. 5d shows the pharmacokinetic blood concentration of the antibodies at 2, 24, 44 and 96 hours post sporozoite challenge.

Allowing for the differences in the serum concentrations between each mAb a statistical analysis of 356-ALE-LS with 356-LS shows that the effect of Fc enhancement is very small with probabilities that 356-ALE-LS is at least 1 fold more potent than 356-LS of near or above 90% (87.60% day 2, 99.97% day 3, 94.96% day 5). These probabilities fell to 21.06% (day 2), 63.05% (day 3) and 5.55% (day 5) for a three-fold change.

FIG. 6 In vitro evaluation of CSP mediated activation of primary NK cells isolated from both wild-type C57BL/6 mice and mice transgenic for human Fcγ receptors. Recombinant CSP protein was coated onto microtitre plates followed by incubation with anti-CSP antibodies, then addition of NK cells isolated from spleens of wild-type C57BL/6 mice or transgenic mice expressing human FcγRs and incubation for three hours. NK activation was assessed by staining for CD107a.

FIG. 7 Quantification of parasites in peripheral blood at day 3 post-sporozoite challenge was measured by real-time PCR. Passive transfer (100 μg (left) and 25 μg (right)) of the indicated mAbs (EpoFix as an isotype control, 356-LS, 356-ALE-LS, 356-LAGA-LS) in WT C57BL/6 and human FcγR mice was administered 24 hours before the challenge with 1500 sporozoites.

FIG. 8 In vivo neutralizing activity of 356-ALE-LS and L9-LS against P. falciparum in FRG-huHep. (A) Representative images of parasite liver burden load measured by bioluminescence of luciferase-expressing transgenic P. falciparum parasites. (B) Liver burden reduction (total flux, photons/sec) 6 days post-infection. (C) Protection data at day 8 post-sporozoite challenge. (D) Serum antibody concentration (g/mL) at the time of the parasite challenge.

### DETAILED DESCRIPTION OF THE INVENTION

In one aspect of the invention there is provided a Plasmodium falciparum circumsporozoite protein (CSP) antigen binding protein.

In another aspect there is provided a circumsporozoite protein (CSP) antigen binding protein comprising: CDRH1 of SEQ ID NO: 1, CDRH2 of SEQ ID NO: 2, CDRH3 of SEQ ID NO: 3, CDRL1 of SEQ ID NO: 4, CDRL2 of SEQ ID NO: 5; and CDRL3 of SEQ ID NO: 6 and wherein the antigen binding protein is modified to have increased effector function and increased half life.

In another aspect there is provided a circumsporozoite protein (CSP) antigen binding protein comprising: CDRH1 of SEQ ID NO: 11, CDRH2 of SEQ ID NO: 12, CDRH3 of SEQ ID NO: 13, CDRL1 of SEQ ID NO: 14, CDRL2 of SEQ ID NO: 15; and CDRL3 of SEQ ID NO: 16 and wherein the antigen binding protein is modified to have increased effector function or increased half life.

In another aspect there is provided a circumsporozoite protein (CSP) antigen binding protein comprising: CDRH1 of SEQ ID NO: 11, CDRH2 of SEQ ID NO: 12, CDRH3 of SEQ ID NO: 13, CDRL1 of SEQ ID NO: 14, CDRL2 of SEQ ID NO: 15; and CDRL3 of SEQ ID NO: 16 and wherein the antigen binding protein is modified to have increased effector function and increased half life.

In another aspect there is provided a circumsporozoite protein (CSP) antigen binding protein comprising: CDRH1 of SEQ ID NO: 1, CDRH2 of SEQ ID NO: 2, CDRH3 of SEQ ID NO: 3, CDRL1 of SEQ ID NO: 4, CDRL2 of SEQ ID NO: 5; and CDRL3 of SEQ ID NO: 6 and wherein the antigen binding protein is modified to have increased effector function or increased half life.

In another aspect there is provided a circumsporozoite protein (CSP) antigen binding protein comprising: CDRH1 of SEQ ID NO: 11, CDRH2 of SEQ ID NO: 12, CDRH3 of SEQ ID NO: 13, CDRL1 of SEQ ID NO: 14, CDRL2 of SEQ ID NO: 15; and CDRL3 of SEQ ID NO: 16 and wherein the antigen binding protein is modified to have increased effector function or increased half life.

In another aspect there is provided a circumsporozoite protein (CSP) antigen binding protein comprising: CDRH1 of SEQ ID NO: 1, CDRH2 of SEQ ID NO: 2, CDRH3 of SEQ ID NO: 3, CDRL1 of SEQ ID NO: 4, CDRL2 of SEQ ID NO: 5; and CDRL3 of SEQ ID NO: 6 and wherein the antigen binding protein is mutated at at least one of the following positions or combinations of positions:
i) S239D
ii) I332E
iii) S239D/I332E
iv) S239D/I332E/A330L
v) G236A/I332E
vi) S239D/I332E/G236A
vii) G236A/S239D/I332E/A330L
viii) H268F/S324T/G236A/I332E
ix) S267E/H268F/S324T/G236A/I332E
x) S298A/E333A/K334A
xi) F243L/R292P/Y300L/P396L
xii) F243L/R292P/Y300L/V305I/P396L
xiii) L235V/F243L/R292P/Y300L/P396L
xiv) F243L/R292P/Y300L
xv) G236A/A330L/I332E
xvi) K228M/K334E
xvii) S239D/I332E/G236A/T250V/A287F
xviii) H268F/S324T/S239D/I332E
xix) T250V/A287F/G236A/A330L/I332E
xx) T250V/A287F
   and wherein the antigen binding protein has increased effector function.

In another aspect there is provided a circumsporozoite protein (CSP) antigen binding protein comprising: CDRH1 of SEQ ID NO: 11, CDRH2 of SEQ ID NO: 12, CDRH3 of SEQ ID NO: 13, CDRL1 of SEQ ID NO: 14, CDRL2 of SEQ ID NO: 15; and CDRL3 of SEQ ID NO: 16 and wherein the antigen binding protein is mutated at at least one of the following positions or combinations of positions:
i) S239D
ii) I332E
iii) S239D/I332E
iv) S239D/I332E/A330L
v) G236A/I332E
vi) S239D/I332E/G236A
vii) G236A/S239D/I332E/A330L
viii) H268F/S324T/G236A/I332E
ix) S267E/H268F/S324T/G236A/I332E
x) S298A/E333A/K334A
xi) F243L/R292P/Y300L/P396L
xii) F243L/R292P/Y300L/V305I/P396L
xiii) L235V/F243L/R292P/Y300L/P396L
xiv) F243L/R292P/Y300L
xv) G236A/A330L/I332E
xvi) K228M/K334E
xvii) S239D, I332E, G236A, T250V, A287F
xviii) H268F/S324T/S239D/I332E
xix) T250V/A287F/G236A/A330L/I332E
xx) T250V/A287F
and wherein the antigen binding protein has increased effector function.

It is known from studies in naturally infected human patients that antibody effector function can develop over time and that this correlates with improved protection. However, this only occurs after multiple mosquito bite challenges with P. falciparum and can take many years to develop (Feng et al, 2021, Nat Comms 12:1742). There are currently no direct acting anti-malaria antibodies that have been shown to have an Fc-mediated role in protection, moreover none that have been further modified in order to improve Fc-mediated host effector cell mechanisms.

Due to the difficulty in showing cell killing of parasite and parasite infected cells by targeting the CSP (which is not naturally expressed in human cells in the absence of infection) the present inventors have shown improved anti-malarial activity by showing improved antibody-dependent NK cell activation (ADNKA) and Antibody-Dependent Cellular Phagocytosis (ADCP) effector activity by modifying the Fc region and this acts as a marker for increased antibody-mediated effector function or ADCC. The use of a mAb with such improved effector function could therefore provide this additional protective mechanism at the earliest possible stage especially in young children enabling more effective prophylaxis.

An additional advantage of the antigen binding proteins of the present invention is the formation of immune-complexes (IC's). The antigen binding protein when bound to infectious parasites can engage the host adaptive immune response by binding to antigen presenting cells particularly dendritic cells (DC's) and, by subsequent antigen presentation result in the priming of T-cell and B-cell immune responses that target the infecting antigen. In this way a ‘vaccine-like’ response to the infectious agent can be evoked providing improved longer-term immune protection. This would bring additional benefit to the near term immuno-prophylactic direct acting Fab and Fc effector function mechanisms described above. The Fc-enhanced antigen binding proteins, for example mAbs of the present invention may have a more pronounced effect by providing improved targeting of antigen-presenting cells and thus improved engagement of the adaptive host immune response.

In another aspect there is provided a circumsporozoite protein (CSP) antigen binding protein comprising: CDRH1 of SEQ ID NO: 1, CDRH2 of SEQ ID NO: 2, CDRH3 of SEQ ID NO: 3, CDRL1 of SEQ ID NO: 4, CDRL2 of SEQ ID NO: 5; and CDRL3 of SEQ ID NO: 6 and wherein the antigen binding protein is modified to have increased half life.

In another aspect there is provided a circumsporozoite protein (CSP) antigen binding protein comprising: CDRH1 of SEQ ID NO: 11, CDRH2 of SEQ ID NO: 12, CDRH3 of SEQ ID NO: 13, CDRL1 of SEQ ID NO: 14, CDRL2 of SEQ ID NO: 15; and CDRL3 of SEQ ID NO: 16 and wherein the antigen binding protein is modified to have increased half life.

As used herein the term increased half life refers to an increase in the time required for the serum concentration of an antigen binding protein to reach half of its original value relative to a wild type antigen binding protein in particular an IgG1 antibody that does not contain modifications to its Fc region when measured in an FcRn binding assay.

A number of mechanisms are described throughout to increase half life and are considered aspects of the invention as herein described. Amino acid substitutions to enable extended serum half life were added in addition to those amino acid substitutions enabling improved ADNKA and ADCP because it is desirable to maintain the serum concentration of a malaria mAb for an extended period of time to minimise antibody administration in malaria endemic countries to cover seasonal malaria.

In another aspect there is provided a circumsporozoite protein (CSP) antigen binding protein comprising: CDRH1 of SEQ ID NO: 1, CDRH2 of SEQ ID NO: 2, CDRH3 of SEQ ID NO: 3, CDRL1 of SEQ ID NO: 4, CDRL2 of SEQ ID NO: 5; and CDRL3 of SEQ ID NO: 6 and wherein the antigen binding protein is mutated at one of the following combinations of positions:
M428L and N434S
M252Y, S254T and T256E
H433K and N434F
and wherein the antigen binding protein has increased half life.

In another aspect there is provided a circumsporozoite protein (CSP) antigen binding protein comprising: CDRH1 of SEQ ID NO: 11, CDRH2 of SEQ ID NO: 12, CDRH3 of SEQ ID NO: 13, CDRL1 of SEQ ID NO: 14, CDRL2 of SEQ ID NO: 15; and CDRL3 of SEQ ID NO: 16 and wherein the antigen binding protein is mutated at one of the following combinations of positions:
M428L and N434S
M252Y, S254T and T256E
H433K and N434F
and wherein the antigen binding protein has increased half life.

In another aspect there is provided a circumsporozoite protein (CSP) antigen binding protein comprising: CDRH1 of SEQ ID NO: 1, CDRH2 of SEQ ID NO: 2, CDRH3 of SEQ ID NO: 3, CDRL1 of SEQ ID NO: 4, CDRL2 of SEQ ID NO: 5; and CDRL3 of SEQ ID NO: 6 and wherein the antigen binding protein is modified to have increased effector function and increased half life.

In another aspect there is provided a circumsporozoite protein (CSP) antigen binding protein comprising: CDRH1 of SEQ ID NO: 11, CDRH2 of SEQ ID NO: 12, CDRH3 of SEQ ID NO: 13, CDRL1 of SEQ ID NO: 14, CDRL2 of SEQ ID NO: 15; and CDRL3 of SEQ ID NO: 16 and wherein the antigen binding protein is modified to have increased effector function and increased half life.

In another aspect there is provided a circumsporozoite protein (CSP) antigen binding protein comprising: CDRH1 of SEQ ID NO: 1, CDRH2 of SEQ ID NO: 2, CDRH3 of SEQ ID NO: 3, CDRL1 of SEQ ID NO: 4, CDRL2 of SEQ ID NO: 5; and CDRL3 of SEQ ID NO: 6 and wherein the antigen binding protein comprises the mutations G236A, A330L, I332E, M428L and N434S.

In another aspect there is provided a circumsporozoite protein (CSP) antigen binding protein comprising: CDRH1 of SEQ ID NO: 11, CDRH2 of SEQ ID NO: 12, CDRH3 of SEQ ID NO: 13, CDRL1 of SEQ ID NO: 14, CDRL2 of SEQ ID NO: 15; and CDRL3 of SEQ ID NO: 16 and wherein the antigen binding protein comprises the mutations G236A, A330L, I332E, M428L and N434S.

In another aspect there is provided a circumsporozoite protein (CSP) antigen binding protein comprising: CDRH1 of SEQ ID NO: 1, CDRH2 of SEQ ID NO: 2, CDRH3 of SEQ ID NO: 3, CDRL1 of SEQ ID NO: 4, CDRL2 of SEQ ID NO: 5; and CDRL3 of SEQ ID NO: 6 and wherein the antigen binding protein comprises the mutations S239D, I332E, G236A, T250V, A287F, M428L and N434S.

In another aspect there is provided a circumsporozoite protein (CSP) antigen binding protein comprising: CDRH1 of SEQ ID NO: 11, CDRH2 of SEQ ID NO: 12, CDRH3 of SEQ ID NO: 13, CDRL1 of SEQ ID NO: 14, CDRL2 of SEQ ID NO: 15; and CDRL3 of SEQ ID NO: 16 and wherein the antigen binding protein comprises the mutations S239D, I332E, G236A, T250V, A287F, M428L and N434S.

In another aspect there is provided a circumsporozoite protein (CSP) antigen binding protein comprising a variable heavy chain of SEQ ID NO: 7 and a variable light chain sequence of SEQ ID NO:8 and wherein the antigen binding protein has increased effector function and increased half life.

In another aspect there is provided a circumsporozoite protein (CSP) antigen binding protein comprising a variable heavy chain of SEQ ID NO: 7 and a variable light chain sequence of SEQ ID NO:8 and wherein the antigen binding protein is mutated in positions G236A, A330L, I332E, M428L and N434S. In one aspect the antigen binding protein has increased effector function and increased half life.

In another aspect there is provided a circumsporozoite protein (CSP) antigen binding protein comprising a variable heavy chain of SEQ ID NO: 7 and a variable light chain sequence of SEQ ID NO:8 and wherein the antigen binding protein is mutated in positions S239D, I332E, G236A, T250V, A287F, M428L and N434S. In one aspect the antigen binding protein has increased effector function and increased half life.

In another aspect there is provided a circumsporozoite protein (CSP) antigen binding protein comprising a variable heavy chain of SEQ ID NO: 17 and a variable light chain sequence of SEQ ID NO:18 and wherein the antigen binding protein has increased effector function and increased half life.

In another aspect there is provided a circumsporozoite protein (CSP) antigen binding protein comprising a variable heavy chain of SEQ ID NO: 17 and a variable light chain sequence of SEQ ID NO:18 and wherein the antigen binding protein is mutated in positions G236A, A330L, I332E, M428L and N434S. In one aspect the antigen binding protein has increased effector function and increased half life.

In another aspect there is provided a circumsporozoite protein (CSP) antigen binding protein comprising a heavy chain of SEQ ID NO: 9 and a light chain sequence of SEQ ID NO:10.

In one aspect there is provided a nucleic acid sequence encoding the heavy chain of SEQ ID NO:9; and/or a nucleic acid sequence encoding the light chain of SEQ ID NO: 10.

In one aspect there is provided an expression vector or vectors comprising a nucleic acid sequence encoding the heavy chain of SEQ ID NO:9; and/or a nucleic acid sequence encoding the light chain of SEQ ID NO: 10.

In one aspect there is provided a recombinant host cell comprising the nucleic acid sequence(s) encoding the heavy chain of SEQ ID NO:9; and/or a nucleic acid sequence encoding the light chain of SEQ ID NO: 10 or an expression vector or vectors comprising a nucleic acid sequence encoding the heavy chain of SEQ ID NO:9; and/or a nucleic acid sequence encoding the light chain of SEQ ID NO: 10.

In one aspect there is provided a method for the production of a CSP antigen binding protein according to the invention as disclosed herein, which method comprises culturing a host cell under conditions suitable for expression of said nucleic acid sequence(s) or vector(s) according to the invention as herein described, whereby a polypeptide comprising the CSP antigen binding protein is produced.

In one aspect there is provided a pharmaceutical composition comprising the CSP antigen binding protein according to the invention as herein described and a pharmaceutically acceptable excipient.

In one aspect there is provided a method for the prevention of malaria in a subject in need thereof comprising administering to said subject a therapeutically effective amount of the CSP antigen binding protein according to the invention as herein described, or a pharmaceutical composition comprising the CSP antigen binding protein according to the invention as herein described.

In one aspect the CSP antigen binding protein according to the invention as herein described or a pharmaceutical composition comprising the CSP antigen binding protein according to the invention as herein described is for use in the prophylaxis of malaria.

In one aspect there is provided the use of a CSP binding protein according to the invention as herein described or a pharmaceutical composition comprising the CSP binding protein according to the invention as herein described, in the manufacture of a medicament for use in the prophylaxis of malaria.

The term CSP binding protein as used herein refers to antibodies and other protein constructs, such as domains, that are capable of binding to Plasmodium falciparum circumsporozoite protein. The terms CSP binding protein and CSP antigen binding protein are used interchangeably herein.

The term antibody is used herein in the broadest sense to refer to molecules with an immunoglobulin-like domain (for example IgG, IgM, IgA, IgD or IgE) and includes monoclonal, recombinant, polyclonal, chimeric, human, humanised, multispecific antibodies, including bispecific antibodies, and heteroconjugate antibodies; antigen binding antibody fragments, Fab, F(ab′)2, Fv, disulphide linked Fv, single chain Fv, disulphide-linked scFv, diabodies, TANDABS, etc. and modified versions of any of the foregoing (for a summary of alternative antibody formats see Holliger and Hudson, 2005, Nature Biotechnology, 23(9):1126-1136).

The term, full, whole or intact antibody, used interchangeably herein, refers to a heterotetrameric glycoprotein with an approximate molecular weight of 150,000 daltons. An intact antibody is composed of two identical heavy chains (HCs) and two identical light chains (LCs) linked by covalent disulphide bonds. This H₂L₂ structure folds to form three functional domains comprising two antigen-binding fragments, known as ‘Fab’ fragments, and a ‘Fc’ crystallisable fragment. The Fab fragment is composed of the variable domain at the amino-terminus, variable heavy (VH) or variable light (VL), and the constant domain at the carboxyl terminus, CH1 (heavy) and CL (light). The Fc fragment is composed of two domains formed by dimerization of paired CH2 and CH3 regions. The Fc may elicit effector functions by binding to receptors on immune cells or by binding C1q, the first component of the classical complement pathway. The five classes of antibodies IgM, IgA, IgG, IgE and IgD are defined by distinct heavy chain amino acid sequences, which are called μ, α, γ, ε and δ respectively, each heavy chain can pair with either a κ or λ light chain. The majority of antibodies in the serum belong to the IgG class, there are four isotypes of human IgG (IgG1, IgG2, IgG3 and IgG4), the sequences of which differ mainly in their hinge region.

In one aspect of the invention as herein descried the CSP antigen binding protein is an antibody. In another aspect it is a monoclonal antibody. In another aspect it is an IgG1 antibody.

Fully human antibodies can be obtained using a variety of methods, for example using yeast-based libraries or transgenic animals (e.g. mice) that are capable of producing repertoires of human antibodies. Yeast presenting human antibodies on their surface that bind to an antigen of interest can be selected using FACS (Fluorescence-Activated Cell Sorting) based methods or by capture on beads using labelled antigens. Transgenic animals that have been modified to express human immunoglobulin genes can be immunised with an antigen of interest and antigen-specific human antibodies isolated using B-cell sorting techniques. Human antibodies produced using these techniques can then be characterised for desired properties such as affinity, developability and selectivity.

Alternative antibody formats include alternative scaffolds in which the one or more CDRs of the antigen binding protein can be arranged onto a suitable non-immunoglobulin protein scaffold or skeleton, such as an affibody, a SpA scaffold, an LDL receptor class A domain, an avimer (see, e.g., U.S. Patent Application Publication Nos. 2005/0053973, 2005/0089932, 2005/0164301) or an EGF domain.

Antigen binding site refers to a site on an antigen binding protein that is capable of specifically binding to an antigen, this may be a single variable domain, or it may be paired VH/VL domains as can be found on a standard antibody. Single-chain Fv (ScFv) domains can also provide antigen-binding sites.

Affinity, also referred to as binding affinity, is the strength of binding at a single interaction site, i.e. of one molecule, e.g. an antigen binding protein of the invention, to another molecule, e.g. its target antigen, at a single binding site. The binding affinity of an antigen binding protein to its target may be determined by equilibrium methods (e.g. enzyme-linked immunoabsorbent assay (ELISA) or radioimmunoassay (RIA)), or kinetics (e.g. surface plasmon resonance, SPR, analysis). For example, the SPR methods described in Example 1 may be used to measure binding affinity.

In one aspect the CSP antigen binding protein binds with an affinity of at least 2.5 nM when measured by Surface Plasmon Resonance (SPR).

In an aspect, the equilibrium dissociation constant (KD) of the antigen binding protein interaction is 3 nM or less. For example the antigen binding protein interaction is 2.5 nM or less or for example is 2 nM or less for example 1 nM or less or for example 100 pM or less or for example is 70 pM or less. In one aspect the antigen binding protein interaction is 45 pM or less. A skilled person will appreciate that the smaller the KD numerical value, the stronger the binding. In one aspect the antigen binding protein interaction is 10 pM to 3 nM or for example 1 nM to 2.5 nM.

The term neutralises as used throughout the present specification means that the biological activity of CSP is reduced in the presence of an antigen binding protein as described herein in comparison to the activity of CSP in the absence of the antigen binding protein, in vitro or in vivo. Neutralisation may be due to one or more of blocking CSP binding to its receptor, preventing CSP from activating its receptor preventing CPS proteolysis, down regulating CSP or its receptor, or affecting effector functionality neutralizing capability of an anti-CSP binding protein.

CDRs are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. Thus, CDRs as used herein refers to all three heavy chain CDRs, all three light chain CDRs, all heavy and light chain CDRs, or at least two CDRs.

Throughout this specification, amino acid residues in variable domain sequences and variable domain regions within full-length antigen binding sequences, e.g. within an antibody heavy chain sequence or antibody light chain sequence, are numbered according to the Kabat numbering convention. Similarly, the terms CDR, CDRL1, CDRL2, CDRL3, CDRH1, CDRH2, CDRH3 used in the Examples follow the Kabat numbering convention. For further information, see Kabat et al., Sequences of Proteins of Immunological Interest, 4th Ed., U.S. Department of Health and Human Services, National Institutes of Health (1987).

It will be apparent to those skilled in the art that there are alternative numbering conventions for amino acid residues in variable domain sequences and full-length antibody sequences. There are also alternative numbering conventions for CDR sequences, for example those set out in Chothia et al, 1989, Nature, 342: 877-883. The structure and protein folding of the antigen binding protein may mean that other residues are considered part of the CDR sequence and would be understood to be so by a skilled person.

Other numbering conventions for CDR sequences available to a skilled person include AbM (University of Bath) and contact (University College London) methods.

Table A below represents one definition using each numbering convention for each CDR or binding unit. The Kabat numbering scheme is used in Table A to number the variable domain amino acid sequence. It should be noted that some of the CDR definitions may vary depending on the individual publication used.

TABLE A

| | Kabat CDR | Chothia CDR | AbM CDR | Contact CDR |
|---|---|---|---|---|
| H1 | 31-35/35A/35B | 26-32/33/34 | 26-35/35A/35B | 30-35/35A/35B |
| H2 | 50-65 | 52-56 | 50-58 | 47-58 |
| H3 | 95-102 | 95-102 | 95-102 | 93-101 |
| L1 | 24-34 | 24-34 | 24-34 | 30-36 |
| L2 | 50-56 | 50-56 | 50-56 | 46-55 |
| L3 | 89-97 | 89-97 | 89-97 | 89-96 |

CDRs may be modified by at least one amino acid substitution, deletion or addition, wherein the variant antigen binding protein substantially retains the biological characteristics of the unmodified protein.

It will be appreciated that each of CDR H1, H2, H3, L1, L2, L3 may be modified alone or in combination with any other CDR, in any permutation or combination. In one embodiment, a CDR is modified by the substitution, deletion or addition of up to 3 amino acids, for example 1 or 2 amino acids, for example 1 amino acid. Typically, the modification is a substitution, particularly a conservative substitution, for example as shown in Table B below.

TABLE B

| Side chain | Members |
|---|---|
| Hydrophobic | Met, Ala, Val, Leu, Ile |
| Neutral hydrophilic | Cys, Ser, Thr |
| Acidic | Asp, Glu |
| Basic | Asn, Gln, His, Lys, Arg |
| Residues that influence chain orientation | Gly, Pro |
| Aromatic | Trp, Tyr, Phe |

For example, in a variant CDR, the flanking residues that comprise the CDR as part of alternative definition(s) e.g. Kabat or Chothia, may be substituted with a conservative amino acid residue.

Such antigen binding proteins comprising variant CDRs as described above may be referred to herein as functional CDR variants.

The numbering used in the mutations or combinations of mutations are done according to standard antibody numbering and would be understood to those skilled in the art.

Neutralisation may be determined or measured using one or more assays known to the skilled person or as described herein.

Percent identity or % identity between a query nucleic acid sequence and a subject nucleic acid sequence is the Identities value, expressed as a percentage, that is calculated using a suitable algorithm (e.g. BLASTN, FASTA, Needleman-Wunsch, Smith-Waterman, LALIGN, or GenePAST/KERR) or software (e.g. DNASTAR Lasergene, GenomeQuest, EMBOSS needle or EMBOSS infoalign), over the entire length of the query sequence after a pair-wise global sequence alignment has been performed using a suitable algorithm (e.g. Needleman-Wunsch or GenePAST/KERR) or software (e.g. DNASTAR Lasergene or GenePAST/KERR). Importantly, a query nucleic acid sequence may be described by a nucleic acid sequence disclosed herein, in particular in one or more of the claims.

Percent identity or % identity between a query amino acid sequence and a subject amino acid sequence is the Identities value, expressed as a percentage, that is calculated using a suitable algorithm (e.g. BLASTP, FASTA, Needleman-Wunsch, Smith-Waterman, LALIGN, or GenePAST/KERR) or software (e.g. DNASTAR Lasergene, GenomeQuest, EMBOSS needle or EMBOSS infoalign), over the entire length of the query sequence after a pair-wise global sequence alignment has been performed using a suitable algorithm (e.g. Needleman-Wunsch or GenePAST/KERR) or software (e.g. DNASTAR Lasergene or GenePAST/KERR). Importantly, a query amino acid sequence may be described by an amino acid sequence disclosed herein, in particular in one or more of the claims.

The query sequence may be 100% identical to the subject sequence, or it may include up to a certain integer number of amino acid or nucleotide alterations as compared to the subject sequence such that the % identity is less than 100%. For example, the query sequence is at least 50, 60, 70, 75, 80, 85, 90, 95, 96, 97, 98, or 99% identical to the subject sequence. In the case of nucleic acid sequences, such alterations include at least one nucleotide residue deletion, substitution or insertion, wherein said alterations may occur at the 5′- or 3′-terminal positions of the query sequence or anywhere between those terminal positions, interspersed either individually among the nucleotide residues in the query sequence or in one or more contiguous groups within the query sequence. In the case of amino acid sequences, such alterations include at least one amino acid residue deletion, substitution (including conservative and non-conservative substitutions), or insertion, wherein said alterations may occur at the amino- or carboxy-terminal positions of the query sequence or anywhere between those terminal positions, interspersed either individually among the amino acid residues in the query sequence or in one or more contiguous groups within the query sequence.

For antibody sequences, the % identity may be determined across the entire length of the query sequence, including the CDRs. Alternatively, the % identity may exclude one or more or all of the CDRs, for example all of the CDRs are 100% identical to the subject sequence and the % identity variation is in the remaining portion of the query sequence, e.g. the framework sequence, so that the CDR sequences are fixed and intact. The variant sequence substantially retains the biological characteristics of the unmodified protein.

The term Effector Function as used herein refers to one or more of antibody-mediated effects including antibody dependent NK activation (ADNKA), antibody-dependent cell-mediated cytotoxicity (ADCC), antibody-mediated complement activation including complement-dependent cytotoxicity (CDC), antibody dependent complement-mediated cell lysis (ADCML), and Fc-mediated phagocytosis or antibody-dependent cellular phagocytosis (ADCP) by effector cells including macrophages and neutrophils.

In a further aspect effector function as used throughout the specification is intended to refer to one or more of antibody dependent NK activation (ADNKA), antibody-dependent cell-mediated cytotoxicity (ADCC), or antibody-dependent cellular phagocytosis (ADCP). In a further aspect the effector function is ADCC.

The interaction between the Fc region of an antigen binding protein or antibody and various Fc receptors (FcR) and complement factors, including FcγRI (CD64), FcγRII (CD32), FcγRIII (CD16), FcRn, C1q, and type II Fc receptors is believed to mediate the effector functions of the antigen binding protein or antibody. Significant biological effects can be a consequence of effector functionality. Usually, the ability to mediate effector function requires binding of the antigen binding protein or antibody to an antigen and not all antigen binding proteins or antibodies will mediate every effector function.

Effector function can be assessed in a number of ways including, for example, evaluating ADCC effector function of antibody coated to target cells mediated by Natural Killer (NK) cells via FcγRIIIa, or ADCP effector function mediated by monocytes/macrophages or neutrophils via FcγRIIa, or evaluating CDC effector function of antibody coated to target cells mediated by complement cascade via C1q. For example, an antigen binding protein of the present invention can be assessed for ADCC effector function in a Natural Killer cell activation assay. Examples of such assays can be found in Shields et al, 2001, J Biol Chem, 276:6591-6604; Lazar et al, 2006, PNAS, 103; 4005-4010.

Throughout this specification, amino acid residues in Fc regions, in antibody sequences or full-length antigen binding protein sequences, are numbered according to the EU index numbering convention.

Human IgG1 constant regions containing specific mutations have been shown to enhance binding to Fc receptors. In some cases these mutations have also been shown to enhance effector functions, such as ADCC, ADCP and CDC, as described below. Antigen binding proteins of the present invention may include any of the following mutations.

Enhanced CDC: Fc engineering can be used to enhance complement-based effector function. For example (with reference to IgG1), K326W/E333S; S267E/H268F/S324T and IgG1/IgG3 cross subclass can increase C1q binding; the single E345R and triple E345R/E430G/S440Y mutations result in preformed IgG hexamers (Diebolder et al., Science 2014; 343: 1260-1263; Wang et al, 2018, Protein Cell, 9(1): 63-73).

Enhanced ADCC: Fc engineering can be used to enhance ADCC. For example (with reference to IgG1), F243L/R292P/Y300L/V305I/P396L; S239D/I332E; and S298A/E333A/K334A increase FcγRIIIa binding; S239D/I332E/A330L increases FcγRIIIa binding and decreases FcγRIIb binding; An asymmetric Fc in which one heavy chain contains L234Y/L235Q/G236W/S239M/H268D/D270E/S298A mutations and D270E/K326D/A330M/K334E in the opposing heavy chain, increases affinity for FcγRIIIa F158 (a lower-affinity allele) and FcγRIIIa V158 (a higher-affinity allele) with no increased binding affinity to inhibitory FcγRIIb (Wang et al, 2018, Protein Cell, 9(1): 63-7).

Enhanced ADCP: Fc engineering can be used to enhance ADCP. For example (with reference to IgG1), G236A/S239D/I332E increases FcγRIIa binding and increases FcγRIIIa binding (Richards et al, Mol Cancer Ther, 2008, 7:2517-2527). G236A/S239D/I332E improves binding to FcγRIIa, improves the FcγRIIa/FcγRIIb binding ratio (activating/inhibitory ratio), and enhances phagocytosis of antibody-coated target cells by macrophages (Wang et al, 2018, Protein Cell, 9(1): 63-7).

### Glycosylation

An antigen binding protein of the present invention may comprise a heavy chain constant region with an altered glycosylation profile, such that the antigen binding protein has an enhanced effector function, e.g. enhanced ADCC, enhanced CDC, enhanced ADCP, or combinations thereof. Examples of suitable methodologies to produce antigen binding proteins with an altered glycosylation profile are described in WO2003011878, WO2006014679 and EP1229125, all of which can be applied to the antigen binding proteins of the present invention.

The absence of the α1,6 innermost fucose residues on the Fc glycan moiety on N297 of IgG1 antibodies enhances affinity for FcγRIIIA. As such, afucosylated or low fucosylated monoclonal antibodies may have increased therapeutic efficacy (Shields et al, J Biol Chem, 2002, 277(30): 26733-40; Monnet et al, 2014, mAbs, 6(2):422-436).

### Complegent

In one embodiment of the present invention there is provided an antigen binding protein comprising a chimeric heavy chain constant region. In an embodiment, the antigen binding protein comprises an IgG1/IgG3 chimeric heavy chain constant region, such that the antigen binding protein has enhanced CDC. For example, a chimeric antigen binding protein of the invention may comprise at least one CH2 domain from IgG3. In one such embodiment, the antigen binding protein comprises one CH2 domain from IgG3 or both CH2 domains may be from IgG3. In an embodiment, the chimeric antigen binding protein comprises an IgG1 CH1 domain, an IgG3 CH2 domain, and an IgG3 CH3 domain. In an embodiment, the chimeric antigen binding protein comprises an IgG1 CH1 domain, an IgG3 CH2 domain, and an IgG3 CH3 domain except for position 435 that is a H (histidine).

In an embodiment, the antigen binding protein comprises an IgG1 CH1 domain and at least one CH2 domain from IgG3. In an embodiment, the chimeric antigen binding protein comprises an IgG1 CH1 domain and the following residues, which correspond to IgG3 residues, in a CH2 domain: 274Q, 276K, 296F, 300F and 339T. In an embodiment, the chimeric antigen binding protein also comprises 356E, which corresponds to an IgG3 residue, within a CH3 domain. In an embodiment, the antigen binding protein also comprises one or more of the following residues, which correspond to IgG3 residues within a CH3 domain: 358M, 384S, 392N, 397M, 4221, 435R, and 436F.

Such methods for the production of antigen binding proteins with chimeric heavy chain constant regions can be performed, for example, using the COMPLEGENT technology system available from BioWa, Inc. (Princeton, NJ) and Kyowa Hakko Kirin Co., Ltd. The COMPLEGENT system comprises a recombinant host cell comprising an expression vector in which a nucleic acid sequence encoding a chimeric Fc region having both IgG1 and IgG3 Fc region amino acid residues is expressed to produce an antigen binding protein having enhanced CDC activity, i.e. CDC activity is increased relative to an otherwise identical antigen binding protein lacking such a chimeric Fc region, as described in WO2007011041 and US20070148165, each of which are incorporated herein by reference. In an alternative embodiment, CDC activity may be increased by introducing sequence specific mutations into the Fc region of an IgG chain. Those of ordinary skill in the art will also recognize other appropriate systems.

### Potelligent

Such methods for the production of antigen binding proteins can be performed, for example, using the POTELLIGENT technology system available from BioWa, Inc. (Princeton, NJ) in which CHOK1SV cells lacking a functional copy of the FUT8 gene produce monoclonal antibodies having enhanced ADCC activity that is increased relative to an identical monoclonal antibody produced in a cell with a functional FUT8 gene as described in U.S. Pat. Nos. 7,214,775, 6,946,292, WO0061739 and WO0231240, all of which are incorporated herein by reference. Those of ordinary skill in the art will also recognize other appropriate systems.

In an embodiment of the invention, the antigen binding protein is produced in a host cell in which the FUT8 gene has been inactivated. In an embodiment of the invention, the antigen binding protein is produced in a −/− FUT8 host cell. In an embodiment of the invention, the antigen binding protein is afucosylated at Asn297 (IgG1).

It will be apparent to those skilled in the art that such modifications may not only be used alone but may be used in combination with each other in order to further enhance effector function.

The half-life of any antigen binding protein refers to the time required for the serum concentration of an antigen binding protein to reach half of its original value. The serum half-life of proteins can be measured by pharmacokinetic studies according to the method described by Kim et al, 1994, Eur J Immunol 24: 542-548. According to this method, radio-labelled protein is injected intravenously into mice and its plasma concentration is periodically measured as a function of time, for example, at about 3 minutes to about 72 hours after the injection. Other methods for pharmacokinetic analysis and determination of the half-life of a molecule will be familiar to those skilled in the art.

The long half-life of IgG antibodies is reported to be dependent on their binding to FcRn. Therefore, substitutions that increase the binding affinity of IgG to FcRn at pH 6.0 while maintaining the pH dependence of the interaction with target, by engineering the constant region, have been extensively studied (Ghetie et al, 1997, Nature Biotech, 15: 637-640; Hinton et al, 2004, J Biol Chem, 279: 6213-6216; Dall'Acqua et al, 2002, J Immunol 169(9):5171-5180). The in-vivo half-life of antigen binding proteins of the present invention may be altered by modification of a heavy chain constant domain or an FcRn binding domain therein.

In adult mammals, FcRn, also known as the neonatal Fc receptor, plays a key role in maintaining serum antibody levels by acting as a protective receptor that binds and salvages antibodies of the IgG isotype from degradation. IgG molecules are endocytosed by endothelial cells and, if they bind to FcRn, are recycled out of the cells back into circulation. In contrast, IgG molecules that enter the cells and do not bind to FcRn are targeted to the lysosomal pathway where they are degraded.

FcRn is believed to be involved in both antibody clearance and the transcytosis across tissues (see Junghans, 1997, Immunol Res, 16:29-57 and Ghetie and Ward, 2000, Annu Rev Immunol 18:739-766). Human IgG1 residues determined to interact directly with human FcRn include Ile253, Ser254, Lys288, Thr307, Gln311, Asn434 and His435. Mutations at any of these positions may enable increased serum half-life and/or altered effector properties of antigen binding proteins of the invention.

Antigen binding proteins of the present invention may have amino acid modifications that increase the affinity of the constant domain or fragment thereof for FcRn. Increasing the half-life (i.e., serum half-life) of therapeutic and diagnostic IgG antibodies and other bioactive molecules has many benefits including reducing the amount and/or frequency of dosing of these molecules. In one embodiment, an antigen binding protein of the invention comprises all or a portion (an FcRn binding portion) of an IgG constant domain having one or more of the following amino acid modifications.

For example, with reference to IgG1, M252Y/S254T/T256E (commonly referred to as YTE mutations) and M428L/N434S (commonly referred to as LS mutations) increase FcRn binding at pH 6.0 (Wang et al, 2018, Protein Cell, 9(1): 63-73).

Half-life can also be enhanced by T250Q/M428L, V259I/V308F/M428L, N434A, and T307A/E380A/N434A mutations (with reference to IgG1 and Kabat numbering) (Monnet et al, 2014, Mabs, 6(2):422-436).

Half-life and FcRn binding can also be extended by introducing H433K and N434F mutations (commonly referred to as HN or NHance mutations) (with reference to IgG1) (WO2006/130834).

WO00/42072 discloses a polypeptide comprising a variant Fc region with altered FcRn binding affinity, which polypeptide comprises an amino acid modification at any one or more of amino acid positions 238, 252, 253, 254, 255, 256, 265, 272, 286, 288, 303, 305, 307, 309, 311, 312, mAb A, 340, 356, 360, 362, 376, 378, 380, 386,388, 400, 413, 415, 424, 433, 434, 435, 436, 439, and 447 of the Fc region (EU index numbering).

WO02/060919 discloses a modified IgG comprising an IgG constant domain comprising one or more amino acid modifications relative to a wild-type IgG constant domain, wherein the modified IgG has an increased half-life compared to the half-life of an IgG having the wild-type IgG constant domain, and wherein the one or more amino acid modifications are at one or more of positions 251, 253, 255, 285-290, 308-314, 385-389, and 428-435.

Shields et al, 2001, J Biol Chem, 276:6591-6604) used alanine scanning mutagenesis to alter residues in the Fc region of a human IgG1 antibody and then assessed the binding to human FcRn. Positions that effectively abrogated binding to FcRn when changed to alanine include 1253, S254, H435, and Y436. Other positions showed a less pronounced reduction in binding as follows: E233-G236, R255, K288, L309, S415, and H433. Several amino acid positions exhibited an improvement in FcRn binding when changed to alanine; notable among these are P238, T256, E272, V305, T307, Q311, D312, K317, D376, E380, E382, S424, and N434. Many other amino acid positions exhibited a slight improvement (D265, N286, V303, K360, Q362, and A378) or no change (S239, K246, K248, D249, M252, E258, T260, S267, H268, S269, D270, K274, N276, Y278, D280, V282, E283, H285, T289, K290, R292, E293, E294, Q295, Y296, N297, S298, R301, N315, E318, K320, K322, S324, K326, A327, P329, P331, E333, K334, T335, S337, K338, K340, Q342, R344, E345, Q345, Q347, R356, M358, T359, K360, N361, Y373, S375, S383, N384, Q386, E388, N389, N390, K392, L398, S400, D401, K414, R416, Q418, Q419, N421, V422, E430, T437, K439, S440, S442, S444, and K447) in FcRn binding.

The most pronounced effect with respect to improved FcRn binding was found for combination variants. At pH 6.0, the E380A/N434A variant showed over 8-fold better binding to FcRn, relative to native IgG1, compared with 2-fold for E380A and 3.5-fold for N434A. Adding T307A to this resulted in a 12-fold improvement in binding relative to native IgG1. In one embodiment the antigen binding protein of the invention comprises the E380A/N434A mutations and has increased binding to FcRn.

Dall'Acqua et al, 2002, J Immunol 169(9):5171-5180 describes random mutagenesis and screening of human IgG1 hinge-Fc fragment phage display libraries against mouse FcRn. They disclosed random mutagenesis of positions 251, 252, 254-256, 308, 309, 311, 312, 314, 385-387, 389, 428, 433, 434, and 436. The major improvements in IgG1-human FcRn complex stability occur when substituting residues located in a band across the Fc-FcRn interface (M252, S254, T256, H433, N434, and Y436) and to lesser extent substitutions of residues at the periphery, such as V308, L309, Q311, G385, Q386, P387, and N389. The variant with the highest affinity to human FcRn was obtained by combining the M252Y/S254T/T256E (YTE) and H433K/N434F/Y436H mutations and exhibited a 57-fold increase in affinity relative to the wild-type IgG1. The in vivo behaviour of such a mutated human IgG1 exhibited a nearly 4-fold increase in serum half-life in cynomolgus monkey as compared to wild-type IgG1.

The present invention therefore provides an antigen binding protein with optimized binding to FcRn. In a preferred embodiment, the antigen binding protein comprises at least one amino acid modification in the Fc region of said antigen binding protein, wherein said modification is at an amino acid position selected from the group consisting of 226, 227, 228, 230, 231, 233, 234, 236, 239, 241, 243, 246, 250, 252, 256, 259, 264, 265, 267, 269, 270, 276, 284, 285, 287, 288, 289, 290, 291, 292, 294, 297, 298, 299, 301, 302, 303, 305, 307, 308, 309, 311, 315, 317, 320, 322, 325, 327, 330, 332, 334, 335, 338, 340, 342, 343, 345, 347, 350, 352, 354, 355, 356, 359, 360, 361, 362, 369, 370, 371, 375, 378, 380, 382, 384, 385, 386, 387, 389, 390, 392, 393, 394, 395, 396, 397, 398, 399, 400, 401403, 404, 408, 411, 412, 414, 415, 416, 418, 419, 420, 421, 422, 424, 426, 428, 433, 434, 438, 439, 440, 443, 444, 445, 446 and 447 of the Fc region.

Additionally, various publications describe methods for obtaining physiologically active molecules with modified half-lives, either by introducing an FcRn-binding polypeptide into the molecules (WO97/43316, U.S. Pat. Nos. 5,869,046, 5,747,035, WO96/32478 and WO91/14438) or by fusing the molecules with antibodies whose FcRn-binding affinities are preserved, but affinities for other Fc receptors have been greatly reduced (WO99/43713) or fusing with FcRn binding domains of antibodies (WO00/09560, U.S. Pat. No. 4,703,039).

FcRn affinity enhanced Fc variants to improve both antibody cytotoxicity and half-life were identified in screens at pH 6.0. The selected IgG variants can be produced as low fucosylated molecules. The resulting variants show increased serum persistence in hFcRn mice, as well as conserved enhanced ADCC (Monnet et al, 2014, mAbs, 6(2):422-436) Exemplary variants include (with reference to IgG1 and Kabat numbering): P230T/V303A/K322R/N389T/F404L/N434S; P228R/N434S; Q311R/K334R/Q342E/N434Y; C226G/Q386R/N434Y; T307P/N389T/N434Y; P230S/N434S; P230T/V305A/T307A/A378V/L398P/N434S; P23OT/P387S/N434S; P230Q/E269D/N434S; N276S/A378V/N434S; T307A/N315D/A330V/382V/N389T/N434Y; T256N/A378V/S383N/N434Y; N315D/A330V/N361D/A387V/N434Y; V259I/N315D/M428L/N434Y; P230S/N315D/M428L/N434Y; F241L/V264E/T307P/A378V/H433R; T250A/N389K/N434Y; V305A/N315D/A330V/P395A/N434Y; V264E/Q386R/P396L/N434S/K439R; E294del/T307P/N434Y (wherein ‘del’ indicates a deletion).

Although substitutions in the constant region are able to significantly improve the functions of IgG antibodies, substitutions in the strictly conserved constant region have the risk of immunogenicity in humans (De Groot and Martin, 2009, Clin Immunol 131: 189-201) and substitution in the highly diverse variable region sequence might be less immunogenic. Reports concerned with the variable region include engineering the CDR residues to improve binding affinity to the antigen (Rothe et al, 2006, Expert Opin Bioi Ther 6: 177-187; Bostrom et al, 2009, Methods Mol Biol 525: 353-376; Thie et al., Methods Mol Biol 525: 309-322, 2009) and engineering the CDR and framework residues to improve stability (Worn and Pluckthun, 2001, J Mol Biol 305: 989-1010; Ewert et al, 2004, Methods 34: 184-199) and decrease immunogenicity risk (De Groot and Martin, 2009, Clin Immunol 131: 189-201; Jones et al, 2009, Methods Mol Bio 525: 405-423, xiv). As reported, improved affinity to the antigen can be achieved by affinity maturation using the phage or ribosome display of a randomized library.

Improved stability can be rationally obtained from sequence- and structure-based rational design. Decreased immunogenicity risk (deimmunization) can be accomplished by various humanization methodologies and the removal of potential T-cell epitopes, which can be predicted using in silico technologies or anticipated by in vitro assays. Additionally, variable regions have been engineered to lower pI. A longer half-life was observed for these antibodies as compared to wild type antibodies despite comparable FcRn binding. Engineering or selecting antibodies with pH-dependent antigen binding can be used to modify antibody and/or antigen half-life e.g. IgG2 antibody half-life can be shortened if antigen-mediated clearance mechanisms normally degrade the antibody when bound to the antigen. Similarly, the antigen:antibody complex can impact the half-life of the antigen, either by extending half-life by protecting the antigen from the typical degradation processes, or by shortening the half-life via antibody-mediated degradation (target-mediated drug disposition). One embodiment relates to antibodies with higher affinity for antigen at pH 7.4 as compared to endosomal pH (i.e., pH 5.5-6.0) such that the KD ratio at pH 5.5/pH 7.4 or at pH 6.0/pH 7.4 is 2 or more. For example to enhance the pharmacokinetic (PK) and pharmacodynamic (PD) properties of the antibody, it is possible to engineer pH-sensitive binding to the antibody by introducing histidine residues into the CDRs.

Antigen binding proteins as described herein may be incorporated into pharmaceutical compositions for use in the prevention or treatment of the human diseases described herein in particular for the prevention of malaria. In one embodiment, the pharmaceutical composition comprises an antigen binding protein in combination with one or more pharmaceutically acceptable carriers and/or excipients.

In a further aspect, provided herein are pharmaceutical compositions for administration of a CSP antigen binding protein of the present invention to a mammalian subject, preferably a human, who is at risk for malaria, in an amount and according to a schedule sufficient to prevent Plasmodium infection. In another aspect the subject resides in a malaria endemic area of Sub Saharan Africa In one such aspect the human subject is a child under 5 years of age. In another aspect the subject is a pregnant woman.

Such compositions may comprise a CSP antigen binding protein as described herein, or a polynucleotide encoding the antigen binding protein, and a pharmaceutically acceptable diluent or carrier. In some embodiments, a polynucleotide encoding the antigen binding protein may be contained in a plasmid vector for delivery, or a viral vector. In some embodiments, the pharmaceutical composition comprises a therapeutically effective amount of the antigen binding protein. As used herein, a therapeutically effective dose or a therapeutically effective amount refers to an amount sufficient to prevent, cure, or at least partially arrest malaria or symptoms of malaria. A therapeutically effective dose can be determined by monitoring a patient's response to therapy. Typical benchmarks indicative of a therapeutically effective dose include amelioration or prevention of symptoms of malaria in the patient, including, for example, reduction in the number of parasites.

In one aspect the prevention of malaria is the complete prevention of the establishment of liver stage disease and complete absence of blood stage disease.

In one aspect prevention of malaria is measured as a delay in the time to onset of detection of parasites in the blood of a relevant subject for example a child or a pregnant woman in a malaria endemic country for example in sub-Saharan Africa.

In one aspect the antigen binding protein according to the invention provides a reduced risk of malaria infection. The risk of infection is measured in clinical trials as the level of protection over a patient population achieved when compared to placebo. For example analysis may be based on the time to the first P. falciparum infection over a 24-week period. The mean time to first infection for the patient population with placebo is much earlier than the group treated with the mAb. In another example, the proportion of participants completely free of P. falciparum infection over the 24-week period compared with placebo is measured. In one aspect the calculated reduction in risk of infection after administration is at least about 20%, 30%, 40%, 50%, for example 60% such as 65%.

In one aspect the antigen binding protein according to the invention provides a reduced risk of developing clinical symptoms of malaria. In one aspect the risk of clinical disease after receiving, for example at least one dose in a 3 month study interval may be reduced by at least about 20%, 30%, 40%, 50%, for example 60% such as approximately 65%.

In one aspect the malaria is non-severe malaria. In another aspect the malaria is severe.

In one aspect the antigen binding protein provides a reduced risk of developing P. falciparum asexual parasitemia in a subject for example despite the presence of fever above 37.5° C. or a history of fever within 24 hours.

In one aspect the reduced risk of malaria infection and reduced risk of developing clinical symptoms of malaria is assessed a period of 3 months after administration of the antigen binding protein.

In one aspect the CSP binding protein provides protection for a period of at least 3 months after administration.

Clinical malaria is defined herein as the presence of malarial parasites in the blood. In a further example clinical malaria may be defined as a fever greater than or equal to 37.5° C. with an asexual parasitaemia of P. falciparum of 500 present per μL of blood or more (for example with a sensitivity and specificity of greater than 90%).

The definition for severe malaria may, for example include the presence of one or more of the following: severe malaria anaemia (PCV <15%), cerebral malaria (Blantyre coma score <2) or severe disease of other body systems which could include multiple seizures (two or more generalized convulsions in the previous 24 hours), prostration (defined as inability to sit unaided), hypoglycaemia <2.2 mmol/dL or <40 mg/dL), clinically suspected acidosis or circulatory collapse.

Amounts effective for use will depend upon factors such as age, weight, administration route, etc. Single or multiple administrations of the antibody will be dependent on the dosage and frequency as required and tolerated by the patient. In some embodiments, the antibody is administered at a pre-erythrocyte stage of infection in a time frame to prevent hepatocyte infection.

Amounts effective for use will depend upon the severity of the disease and the general state of the patient's health, including other factors such as age, weight, gender, administration route, etc. Single or multiple administrations of the antibody will be dependent on the dosage and frequency as required and tolerated by the patient. In some embodiments, the antibody is administered at a pre-erythrocyte stage of infection or is administered in a time frame to prevent or reduce hepatocyte infection.

In one aspect the antigen binding protein is dosed at least once prior to transmission season, in another aspect the antigen binding protein is dosed at least twice prior to transmission season, in another aspect the antigen binding protein is dosed at least three times.

Pharmaceutical compositions may be administered by injection or continuous infusion (examples include, but are not limited to, intravenous, intraperitoneal, intradermal, subcutaneous, intramuscular, and intraportal). In one embodiment, the composition is suitable for intramuscular administration or subcutaneous administration. In one aspect the administration is intramuscular. In one embodiment the administration is subcutaneous. Pharmaceutical compositions may be suitable for topical administration (which includes, but is not limited to, epicutaneous, inhaled, intranasal or ocular administration) or enteral administration (which includes, but is not limited to, oral, vaginal, or rectal administration).

In one aspect the CSP antigen binding protein according to the invention as herein described or the pharmaceutical composition as herein described is or a pharmaceutical composition comprising the CSP antigen binding protein as herein described is for intravenous, subcutaneous or intramuscular administration. The pharmaceutical composition may be included in a kit containing the antigen binding protein together with other medicaments, and/or with instructions for use. For convenience, the kit may comprise the reagents in predetermined amounts with instructions for use. The kit may also include devices used for administration of the pharmaceutical composition. In a further aspect the kit may comprise a diagnostic for detecting infection

The terms individual, subject and patient are used herein interchangeably. In one embodiment, the subject is an animal. In another embodiment, the subject is a mammal, such as a primate, for example a marmoset or monkey. In another embodiment, the subject is a human.

The antigen binding protein described herein may also be used in methods of prophylaxis or treatment. The antigen binding protein described herein is used in an effective amount for prophylactic or preventative treatment. As used herein, a therapeutically effective dose or a therapeutically effective amount refers to an amount sufficient to prevent, cure, or at least partially arrest malaria or symptoms of malaria.

In one aspect the antigen binding proteins of the invention as described herein are to be administered to a subject in need thereof, in another aspect the antigen binding proteins are to be administered to patients under 5 years of age. In another aspect the antigen binding proteins of the invention as described herein are to be administered to pregnant women.

In one aspect the antigen binding protein provides protection against blood stage Plasmodium falciparum infection for at least 1 month, or at least 3 months, or at least 5 months or at least 8 months or at least 12 months.

Antigen binding proteins may be prepared by any of a number of conventional techniques. For example, antigen binding proteins may be purified from cells that naturally express them (e.g., an antibody can be purified from a hybridoma that produces it) or produced in recombinant expression systems.

A number of different expression systems and purification regimes can be used to generate the antigen binding protein of the invention. Generally, host cells are transformed with a recombinant expression vector encoding the desired antigen binding protein(s). The expression vector may be maintained by the host as a separate genetic element or integrated into the host chromosome depending on the expression system. A wide range of host cells can be employed, including but not limited to Prokaryotes (including Gram negative or Gram-positive bacteria, for example Escherichia coli, Bacilli sp., Pseudomonas sp., Corynebacterium sp., Eukaryotes including yeast (for example Saccharomyces cerevisiae, Pichia pastoris), fungi (for example Aspergils sp.), or higher Eukaryotes including insect cells and cell lines of mammalian origin (for example, CHO, NS0, PER.C6, HEK293, HeLa).

The host cell may be an isolated host cell. The host cell is usually not part of a multicellular organism (e.g., plant or animal). The host cell may be a non-human host cell.

Appropriate cloning and expression vectors for use with bacterial, fungal, yeast, and mammalian host cells are known in the art.

The cells can be cultured under conditions that promote expression of the antigen binding protein using a variety of equipment such as shake flasks, spinner flasks, and bioreactors. The polypeptide is recovered by conventional protein purification procedures. Protein purification procedures typically consist of a series of unit operations comprised of various filtration and chromatographic processes developed to selectively concentrate and isolate the antigen binding protein. The purified antigen binding protein may be formulated in a pharmaceutically acceptable composition.

TABLE C
sequences

| | Protein | Nucleotide |
|---|---|---|
| Description | sequence | sequence |
| 356 CDRH1 | SEQ ID NO: 1 | N/A |
| 356 CDRH2 | SEQ ID NO: 2 | N/A |
| 356 CDRH3 | SEQ ID NO: 3 | N/A |
| 356 CDRL1 | SEQ ID NO: 4 | N/A |
| 356 CDRL2 | SEQ ID NO: 5 | N/A |
| 356 CDRL3 | SEQ ID NO: 6 | N/A |
| 356 Vh | SEQ ID NO: 7 | SEQ ID NO: 21 |
| 356 VI | SEQ ID NO: 8 | SEQ ID NO: 22 |
| 356 ALE LS Heavy chain | SEQ ID NO: 9 | SEQ ID NO: 23 |
| 356 ALE LS Light Chain | SEQ ID NO: 10 | SEQ ID NO: 24 |
| 338 CDRH1 | SEQ ID NO: 11 | N/A |
| 338 CDRH2 | SEQ ID NO: 12 | N/A |
| 338 CDRH3 | SEQ ID NO: 13 | N/A |
| 338 CDRL1 | SEQ ID NO: 14 | N/A |
| 338 CDRL2 | SEQ ID NO: 15 | N/A |
| 338 CDRL3 | SEQ ID NO: 16 | N/A |
| 338 Vh | SEQ ID NO: 17 | SEQ ID NO: 25 |
| 338 VI | SEQ ID NO: 18 | SEQ ID NO: 26 |
| 338 ALE LS Heavy chain | SEQ ID NO: 19 | SEQ ID NO: 27 |
| 338 ALE LS Light Chain | SEQ ID NO: 20 | SEQ ID NO: 28 |
| 356 DEA FV LS heavy chain | SEQ ID NO: 29 | SEQ ID NO: 31 |
| 356 DEA FV LS light chain | SEQ ID NO: 30 | SEQ ID NO: 32 |

Throughout the specification and for the avoidance of doubt the nomenclature: 356-ALE-LS, AB-000356 ALE LS and AB-356 ALE_LS is used interchangeably and relate to the same antibody.

### EXAMPLES

### Example 1 In Vitro and In Vivo Characterisation of a Hit Panel of Malaria mAbs

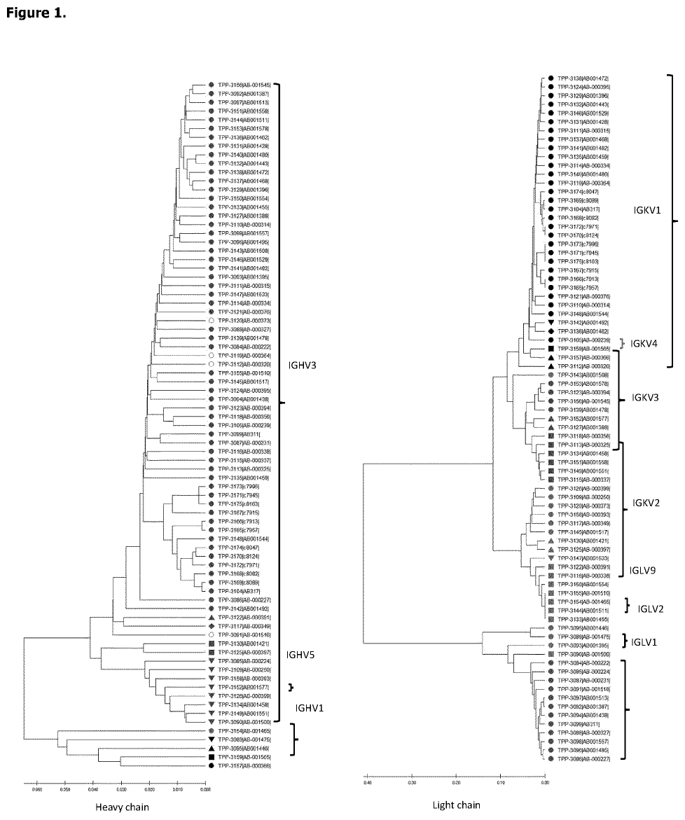

A set of 79 diverse antibody sequences that were originally derived from protected volunteers in an RTS,S phase 2 vaccine trial were selected as a panel for evaluation. The diversity of the hit panel of antibodies are shown in FIG. 1.

Since the developability and manufacturing potential of an antibody does not always align with optimal efficacy using in vitro potency and in vivo pre-clinical disease models the hit panel was first evaluated using in vitro protein analytical assays to determine which antibodies were to be developed further.

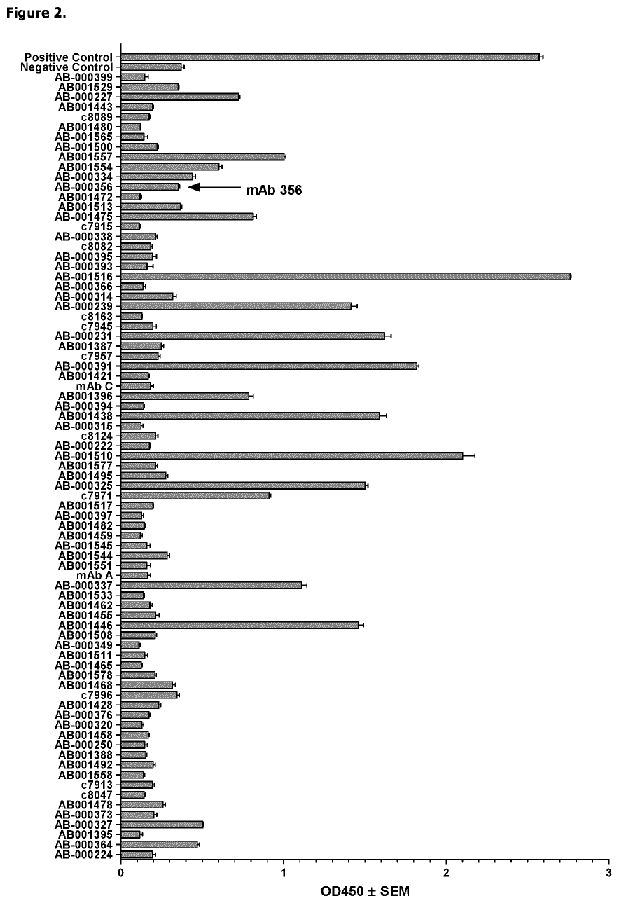

Small scale (125 ml) antibody material generation of the remaining molecules in the hit panel was then undertaken followed by protein analytical testing by size exclusion chromatography (SEC), mass-spectrometry (MS), hydrophobic interaction chromatography (HIC), heparin binding ELISA, nano differential scanning fluorimetry (nano-DSF) assay and, target binding affinity using recombinant CSP by surface plasmon resonance (SPR). Antibodies that did not meet defined criteria were excluded from progression including those that fell below the required purified material yield, those that did not meet the 95% monomer SEC threshold, those that showed binding above positive control in a heparin binding ELISA assay (FIG. 2), antibodies showing a high retention time by HIC (Table 1) and those with a low onset melt profile (Table 2). In addition the SPR data showed several that had no measurable binding or, low binding affinity >3.0 nM (Table 3).

Molecules that were not excluded (designated the lead panel) were then carried forward for efficacy testing in the P. berghei mouse model of malaria sporozoite challenge.

TABLE 1
Hydrophobic Interaction Chromatography data on hit panel
of antibodies All antibodies listed in the table are all
half-life extended with M428L/N434S (LS) modification.

| | | | | Peak | |
|---|---|---|---|---|---|
| | Retention | Peak | Main | Width | |
| Name | time | Area | Area % | 50 perc | Comments |
| Isotype | 1.235 | 1420.814 | 100 | 0.07 | |
| control | | | | | |
| AB001492 | 2.073 | 2628.075 | 84.2 | 0.061 | |
| AB-000349 | 1.659 | 3056.303 | 100 | 0.059 | |
| AB001459 | 1.557 | 3396.671 | 100 | 0.057 | |
| AB-000222 | 1.753 | 2360.837 | 89.08 | 0.061 | |
| AB001387 | 2.4 | 2512.862 | 100 | 0.058 | |
| AB-000395 | 2.233 | 3196.438 | 98.46 | 0.061 | |
| AB-001565 | 1.915 | 1528.929 | 53.31 | 0.056 | multiple |
| | | | | | peaks (3) |
| AB-000224 | 3.123 | 3412.398 | 98.67 | 0.074 | |
| AB001388 | 1.864 | 2962.74 | 100 | 0.083 | |
| AB001508 | 1.301 | 2830.287 | 100 | 0.06 | |
| AB001482 | 1.416 | 3293.539 | 100 | 0.062 | |
| AB-000231 | 1.547 | 1725.44 | 65 | 0.058 | |
| c8082 | 1.7 | 2673.687 | 100 | 0.064 | |
| AB001480 | 1.82 | 2618.721 | 100 | 0.062 | |
| AB-000364 | 1.223 | 3432.539 | 100 | 0.059 | |
| AB-000250 | 1.402 | 2979.951 | 85.51 | 0.069 | |
| AB001446 | 1.59 | 2830.031 | 96.64 | 0.056 | |
| c8124 | 1.537 | 1929.228 | 100 | 0.058 | |
| AB-000338 | 1.54 | 2675.416 | 85.46 | 0.059 | |
| c8089 | 1.737 | 3159.395 | 100 | 0.063 | |
| AB00139523 | 2.118 | 3071.478 | 100 | 0.062 | |
| AB001458 | 1.354 | 2912.357 | 100 | 0.059 | |
| AB001455 | 2.238 | 3716.305 | 97.57 | 0.068 | |
| AB-000397 | 1.21 | 2608.23 | 100 | 0.059 | |
| AB-000315 | 1.494 | 2568.375 | 100 | 0.063 | |
| c7945 | 1.545 | 2911.869 | 100 | 0.066 | |
| c7915 | 1.557 | 2529.881 | 100 | 0.064 | |
| AB001443 | 2.187 | 3108.951 | 100 | 0.073 | |
| AB-000327 | 1.637 | 4072.526 | 100 | 0.064 | |
| mAb B | 1.23 | 3404.848 | 100 | 0.066 | |
| AB001462 | 1.873 | 3243.712 | 100 | 0.06 | |
| AB001517 | 1.374 | 3909.711 | 97.25 | 0.06 | |
| AB001438 | 2.725 | 2705.234 | 95.46 | 0.066 | |
| c8163 | 1.772 | 2482.345 | 100 | 0.062 | |
| AB-001475 | 1.401 | 3335.702 | 97.05 | 0.067 | |
| AB-000227 | 1.635 | 2879.458 | 100 | 0.057 | |
| AB-000373 | 1.657 | 2587.763 | 98.88 | 0.059 | |
| AB-000376 | 1.621 | 3957.999 | 94.9 | 0.063 | |
| AB001533 | 3.076 | 3721.571 | 100 | 0.076 | |
| c7971 | 1.543 | 2749.661 | 100 | 0.065 | |
| AB-000394 | 2.229 | 2207.221 | 97.8 | 0.066 | |
| AB-00023944 | 1.741 | 3236.946 | 98.78 | 0.062 | |
| AB001513 | 2.646 | 3737.497 | 99.01 | 0.069 | |
| AB001529 | 2.366 | 1162.647 | 44.65 | 0.136 | multiple |
| | | | | | peaks (3) |
| AB001478 | 1.14 | 3619.011 | 97.91 | 0.07 | |
| AB001428 | 1.196 | 4224.962 | 100 | 0.065 | |
| AB-000337 | 1.547 | 2974.234 | 97.54 | 0.059 | |
| AB-000325 | 1.292 | 2305.595 | 66.6 | 0.066 | |
| AB001472 | 1.54 | 3566.43 | 100 | 0.059 | |
| AB-000399 | 1.262 | 3007.757 | 95.58 | 0.056 | |
| c8047 | 1.422 | 2962.974 | 100 | 0.063 | |
| c7996 | 1.383 | 2723.425 | 100 | 0.059 | |
| mAb A | 1.753 | 3556.23 | 100 | 0.064 | |
| AB001495 | 2.635 | 4107.609 | 97.51 | 0.068 | |
| AB001396 | 1.955 | 2988.352 | 100 | 0.1 | |
| AB-000314 | 1.886 | 2878.837 | 80.89 | 0.095 | |
| AB-000356 | 1.288 | 2559.599 | 89.21 | 0.06 | |
| c7913 | 1.359 | 4119.076 | 100 | 0.061 | |
| AB001468 | 1.608 | 3679.079 | 100 | 0.073 | |
| AB001551 | 1.523 | 3447.509 | 100 | 0.505 | |
| mAb C | 1.947 | 1406.812 | 100 | 0.068 | |
| AB-000366 | 1.535 | 1694.455 | 44.35 | 0.059 | multiple |
| | | | | | peaks (3) |
| AB-000334 | 1.36 | 2992.896 | 100 | 0.064 | |
| AB001578 | 1.95 | 2718.075 | 100 | 0.067 | |
| AB001577 | 1.308 | 3884.838 | 92.4 | 0.064 | |
| AB001421 | 1.296 | 3201.183 | 100 | 0.057 | |
| AB-001516 | 3.232 | 1854.039 | 96.67 | 0.081 | |
| AB001554 | 2.856 | 2000.126 | 83.73 | 0.077 | |
| AB001558 | 1.1 | 3441.157 | 100 | 0.063 | |
| AB-001465 | 1.128 | 3044.584 | 100 | 0.073 | |
| AB001544 | 1.184 | 3878.29 | 100 | 0.069 | |
| AB-000391 | 0.794 | 975.853 | 45.11 | 0.425 | multiple |
| | | | | | peaks, poor |
| | | | | | shape |
| AB001557 | 1.426 | 2418.161 | 87.91 | 0.06 | |
| AB001511 | 1.925 | 2734.344 | 91.85 | 0.082 | |
| AB-001545 | 1.38 | 1812.905 | 59.23 | 0.068 | |
| AB-001510- | 1.569 | 1662.746 | 72.53 | 0.317 | multiple |
| | | | | | peaks, poor |
| | | | | | shape |
| c7957 | 1.037 | 1460.588 | 58.04 | 0.066 | |
| AB-000393 | 0.998 | 2257.83 | 91.01 | 0.06 | |
| AB-001500 | 1.279 | 2970.758 | 95.14 | 0.064 | |

TABLE 2
Nano-DSF data on the hit panel of antibodies all half-
life extended with M428L/N434S (LS) modification.

| Name | Tm1 | Tm2 | Tm3 |
|---|---|---|---|
| AB-000222 | 66.82 | 75.31 | |
| AB-000224 | 62.79 | 75.53 | |
| AB-000227 | 64.01 | 70.65 | |
| AB-000231 | 64.43 | 75.32 | |
| AB-000239 | 66.44 | 73.47 | 79.18 |
| AB-000250 | 66.25 | 76.39 | |
| AB-000314 | 65.50 | 0.00 | |
| AB-000315 | 67.06 | 74.28 | |
| mAb B | 64.93 | 77.38 | |
| AB-000325 | 66.25 | 72.61 | 77.65 |
| AB-000327 | 64.12 | 76.73 | |
| AB-000334 | 64.39 | 71.72 | 79.06 |
| AB-000337 | 66.41 | 77.28 | 83.26 |
| AB-000338 | 65.56 | 77.62 | |
| AB-000349 | 65.62 | 76.97 | |
| AB-000356 | 68.28 | 0.00 | |
| AB-000364 | 65.33 | 77.27 | |
| AB-000366 | 66.87 | 76.54 | |
| AB-000373 | 63.27 | 77.42 | |
| AB-000376 | 66.80 | 78.10 | |
| AB-000391 | 64.62 | 71.75 | 77.80 |
| AB-000393 | 67.07 | 76.92 | |
| AB-000394 | 64.16 | 77.18 | |
| AB-000395 | 66.30 | 74.95 | |
| AB-000397 | 65.82 | 77.50 | |
| AB-000399 | 66.39 | 79.50 | |
| AB001387 | 66.51 | 76.98 | |
| AB001388 | 65.25 | 77.02 | |
| AB001395 | 63.03 | 76.37 | |
| AB001396 | 60.67 | 69.61 | 78.31 |
| AB001421 | 65.63 | 71.56 | 77.74 |
| AB001428 | 66.24 | 78.02 | |
| AB001438 | 68.09 | 0.00 | |
| AB001443 | 66.06 | 76.79 | |
| AB001446 | 69.21 | 0.00 | |
| AB001455 | 65.04 | 77.43 | |
| AB001458 | 65.46 | 77.64 | |
| AB001459 | 68.12 | 77.83 | |
| AB001462 | 70.01 | 0.00 | |
| AB-001465 | 67.22 | 71.74 | |
| AB001468 | 64.13 | 77.88 | |
| AB001472 | 65.87 | 73.60 | |
| AB-001475 | 66.92 | 76.77 | |
| AB001478 | 66.79 | 74.77 | |
| AB001480 | 64.96 | 72.34 | |
| AB001482 | 65.42 | 77.61 | |
| AB001492 | 66.48 | 77.23 | |
| AB001495 | 67.13 | 73.70 | |
| AB-001500 | 66.46 | 75.54 | 83.75 |
| AB001508 | 66.88 | 73.56 | |
| AB-001510 | 65.36 | 77.62 | |
| AB001511 | 66.84 | 78.19 | |
| AB001513 | 67.73 | 76.58 | |
| AB-001516 | 52.95 | 67.71 | 72.00 |
| AB001517 | 62.61 | 77.20 | |
| AB001529 | 63.22 | 77.99 | |
| AB001533 | 67.84 | 0.00 | |
| AB001544 | 65.16 | 76.99 | |
| AB-001545 | 64.97 | 69.61 | 77.89 |
| AB001551 | 66.08 | 73.82 | |
| AB001554 | 62.00 | 77.18 | |
| AB001557 | 71.63 | 0.00 | |
| AB001558 | 65.41 | 71.11 | 77.50 |
| AB-001565 | 65.69 | 71.62 | |
| AB001577 | 65.77 | 72.78 | |
| AB001578 | 66.46 | 72.38 | |
| mAb C | 66.09 | 76.19 | |
| mAb A | 66.50 | 77.53 | |
| c7913 | 62.71 | 77.35 | |
| c7915 | 64.09 | 76.88 | |
| c7945 | 66.20 | 77.74 | |
| c7957 | 64.93 | 77.40 | |
| c7971 | 65.03 | 71.58 | |
| c7996 | 66.44 | 78.01 | |
| c8047 | 64.73 | 77.13 | |
| c8082 | 66.17 | 77.43 | |
| c8089 | 66.63 | 78.30 | |
| c8124 | 66.59 | 76.43 | |
| c8163 | 64.42 | 76.86 | |

TABLE 3
SPR data using recombinant CSP showing variable binding affinities.
Note that antibodies listed in the table are all half-life
extended with M428L/N434S (LS) modification.

| Name | ka (1/Ms) | kd (1/s) | KD (M) | KD (pM) |
|---|---|---|---|---|
| mAb A | 2.34E+06 | 1.75E−04 | 7.47E−11 | 74.7 |
| AB001492 | 1.32E+07 | 7.27E−04 | 5.51E−11 | 55.1 |
| AB-000349 | 1.19E+06 | 1.72E−03 | 1.44E−09 | 1437.5 |
| AB001459 | 1.12E+07 | 2.02E−03 | 1.81E−10 | 180.9 |
| AB-000222 | 7.76E+06 | 1.18E−03 | 1.53E−10 | 152.5 |
| AB001387 | 1.65E+06 | 1.11E−03 | 6.76E−10 | 675.8 |
| AB-000395 | 1.19E+07 | 2.19E−03 | 1.84E−10 | 183.9 |
| AB-001565 | 1.34E+07 | 6.13E−04 | 4.58E−11 | 45.8 |
| AB-000224 | 8.01E+05 | 3.54E−04 | 4.41E−10 | 441.3 |
| AB001388 | 5.18E+06 | 8.35E−04 | 1.61E−10 | 161.3 |
| AB001508 | 2.31E+06 | 1.40E−03 | 6.04E−10 | 604.4 |
| AB001482 | 3.73E+06 | 9.82E−04 | 2.63E−10 | 263.3 |
| AB-000231 | 4.34E+06 | 1.42E−03 | 3.28E−10 | 328.0 |
| c8082 | 3.04E+06 | 6.85E−04 | 2.25E−10 | 225.5 |
| AB001480 | 5.61E+05 | 6.59E−04 | 1.17E−09 | 1175.0 |
| AB-000364 | 8.87E+06 | 1.26E−03 | 1.42E−10 | 142.3 |
| AB-000250 | 8.13E+05 | 4.87E−04 | 5.99E−10 | 599.4 |
| AB001446 | 4.01E+06 | 7.56E−04 | 1.88E−10 | 188.4 |
| c8124 | 2.22E+06 | 8.00E−04 | 3.61E−10 | 360.9 |
| AB-000338 | 1.13E+07 | 1.47E−03 | 1.30E−10 | 130.2 |
| c8089 | 2.85E+06 | 7.52E−04 | 2.64E−10 | 264.0 |
| AB001395 | 1.33E+06 | 1.54E−03 | 1.16E−09 | 1159.6 |
| AB001458 | 1.86E+06 | 1.71E−03 | 9.21E−10 | 921.1 |
| AB001455 | 6.08E+05 | 8.31E−04 | 1.37E−09 | 1365.9 |
| AB-000397 | 9.17E+05 | 4.30E−04 | 4.69E−10 | 469.3 |
| AB-000315 | 2.01E+07 | 1.25E−03 | 6.22E−11 | 62.2 |
| c7945 | 8.50E+06 | 9.66E−04 | 1.14E−10 | 113.7 |
| c7915 | 3.04E+06 | 6.32E−04 | 2.08E−10 | 207.7 |
| AB001443 | 2.94E+05 | 1.06E−03 | 3.60E−09 | 3602.2 |
| AB-000327 | 3.06E+06 | 1.43E−03 | 4.67E−10 | 467.1 |
| mAb B | 3.88E+06 | 7.74E−04 | 2.00E−10 | 199.7 |
| AB001462 | 3.09E+05 | 1.04E−03 | 3.38E−09 | 3383.6 |
| AB001517 | 1.37E+06 | 1.04E−03 | 7.55E−10 | 755.3 |
| AB001438 | 1.27E+06 | 2.01E−03 | 1.58E−09 | 1583.9 |
| c8163 | 1.18E+07 | 9.01E−04 | 7.67E−11 | 76.7 |
| AB-001475 | 6.41E+05 | 7.09E−04 | 1.11E−09 | 1105.8 |
| AB-000227 | 9.67E+06 | 1.74E−03 | 1.80E−10 | 180.3 |
| AB-000373 | 2.58E+07 | 1.52E−03 | 5.92E−11 | 59.2 |
| AB-000376 | 4.31E+06 | 8.66E−04 | 2.01E−10 | 201.1 |
| AB001533 | 2.69E+06 | 6.69E−04 | 2.48E−10 | 248.5 |
| c7971 | 2.52E+06 | 7.61E−04 | 3.03E−10 | 302.6 |
| AB-000394 | 1.48E+07 | 1.56E−03 | 1.05E−10 | 105.4 |
| AB-000239 | 1.35E+06 | 1.12E−03 | 8.24E−10 | 824.2 |
| AB001513 | 2.40E+06 | 9.31E−04 | 3.88E−10 | 388.1 |
| AB001529 | 6.60E+05 | 1.08E−03 | 1.64E−09 | 1638.6 |
| AB001478 | 1.81E+06 | 1.35E−03 | 7.42E−10 | 742.5 |
| AB001428 | 8.55E+05 | 1.01E−03 | 1.18E−09 | 1177.2 |
| AB-000337 | 4.47E+06 | 8.43E−04 | 1.89E−10 | 188.6 |
| AB-000325 | 4.07E+07 | 2.15E−03 | 5.29E−11 | 52.9 |
| AB001472 | 2.46E+05 | 5.18E−04 | 2.11E−09 | 2107.2 |
| AB-000399 | 9.06E+05 | 6.10E−04 | 6.73E−10 | 672.8 |
| c8047 | 1.82E+06 | 8.08E−04 | 4.44E−10 | 443.7 |
| c7996 | 7.98E+06 | 1.14E−03 | 1.43E−10 | 143.3 |
| mAb A | 2.86E+06 | 5.96E−04 | 2.09E−10 | 208.7 |
| AB001495 | 3.20E+05 | 4.80E−04 | 1.50E−09 | 1498.3 |
| AB001396 | 2.68E+05 | 3.93E−04 | 1.47E−09 | 1468.6 |
| AB-000314 | 2.12E+07 | 1.63E−03 | 7.69E−11 | 76.9 |
| AB-000356 | 4.36E+05 | 1.07E−03 | 2.46E−09 | 2455.2 |
| c7913 | 4.91E+06 | 1.13E−03 | 2.29E−10 | 229.5 |
| AB001468 | 8.58E+05 | 8.12E−04 | 9.46E−10 | 945.9 |
| AB001551 | 2.60E+06 | 1.47E−03 | 5.65E−10 | 565.3 |
| mAb C | 3.66E+06 | 1.49E−03 | 4.08E−10 | 407.9 |
| AB-000366 | 8.50E+06 | 7.04E−04 | 8.27E−11 | 82.7 |
| AB-000334 | 2.61E+05 | 1.10E−03 | 4.21E−09 | 4213.0 |
| AB001578 | 3.69E+05 | 9.77E−04 | 2.64E−09 | 2644.2 |
| AB001577 | 4.15E+06 | 8.30E−04 | 2.00E−10 | 199.9 |
| AB001421 | 1.71E+06 | 6.24E−04 | 3.66E−10 | 365.9 |
| AB-001516 | 1.49E+07 | 1.60E−03 | 1.07E−10 | 107.5 |
| AB001554 | 3.13E+05 | 7.16E−04 | 2.29E−09 | 2285.2 |
| AB001558 | 1.98E+04 | 7.97E−04 | 4.02E−08 | 40211.7 |
| AB-001465 | 1.88E+05 | 5.99E−04 | 3.19E−09 | 3194.0 |
| AB001544 | 3.71E+06 | 9.55E−04 | 2.57E−10 | 257.4 |
| AB-000391 | 8.69E+05 | 5.35E−04 | 6.16E−10 | 615.9 |
| AB001557 | 1.29E+06 | 1.32E−03 | 1.03E−09 | 1028.2 |
| AB001511 | 2.59E+05 | 5.25E−04 | 2.03E−09 | 2028.4 |
| AB-001545 | 8.90E+04 | 4.42E−04 | 4.96E−09 | 4964.4 |
| AB-001510 | n/a | n/a | n/a | n/a |
| c7957 | 4.49E+06 | 1.12E−03 | 2.50E−10 | 250.1 |
| AB-000393 | 8.55E+05 | 7.72E−04 | 9.04E−10 | 903.5 |
| AB-001500 | 8.48E+04 | 6.41E−04 | 7.56E−09 | 7558.8 |

### Example 2 In Vivo Potency Assessment of Lead Panel of Antibodies

The Plasmodium berghei(P. berghei) mouse model of in vivo protection was used to evaluate the protective efficacy of the lead panel. The model comprises C57BL/6 mice infected with a recombinant rodent Plasmodium berghei (P. berghei) strain bearing P. falciparum CSP that also carries a GFP transgene marker to enable visualisation of sporozoite infection of liver cells (Lin et al, 2013, Methods Mol Biol, 923:507-22; Tewari et al, 2002, J Biol Chem, 277(49):47613-8).

As we have previously determined a dose of 300 μg of antibody to be fully protective in this mouse model it was decided that a dose of 200 μg would be selected in order to better evaluate the rank order of antibody efficacy. Animals (n=3) were dosed with each mAb by the i.v route 24 hours prior to administration of 1500 sporozoites. Animals were then examined for liver infection by bio-luminescence at 44 hours, and for blood stage disease by PCR on day 3 and flow cytometry to day 13. The number of animals that were fully protected in each group was evaluated as the primary study end point. A summary of the findings are summarised in Table 4. The results show a wide variation in the level of protection with eight mAbs showing full protection in only 1 animal of 3 (33% protection), another eight mAbs conferred full protection in 2 animals of 3 (66% protection) whilst only 2 mAbs (mAb 356 and 338) conferred 100% full protection at study endpoint day 10. The other mAbs did not show any protection at study end point. Interestingly there is no clear relationship between antibody affinity and in vivo efficacy. The top ten most efficacious molecules were further triaged using a lower dose of 100 μg and the data is summarised in Table 5. Antibodies 356 and 338 were again the most efficacious molecules.

TABLE 4
In vivo evaluation of hit panel antibodies in mouse P. berghei mode.
All antibodies listed in the table are half-life extended with
M428L/N434S (LS) modification except for mAb A.

| | Animals | PK Data | Anti- | |
|---|---|---|---|---|
| | protected | (μg/ml) at | body | Binding |
| | after iv spz | 44 hours | dose | affinity |
| Antibody name | challenge | (Mean ± SD) | (μg) | (KD) |
| mAb A | 13 out of 15 | 61.39 ± 23 | 200 | 74.7 pM |
| AB-000338 | 3 out of 3 | 20.67 ± 5.8 | 200 | 130.2 pM |
| AB-000356 | 3 out of 3 | 64.77 ± 14.3 | 200 | 2455.2 pM |
| AB-000222 | 2 out of 3 | 34.4 ± 3.5 | 200 | 152 pM |
| AB-000334 | 2 out of 3 | 68.87 ± 17.4 | 200 | 4213 pM |
| AB-000394 | 2 out of 3 | 25.17 ± 8.1 | 200 | 105.4 pM |
| AB001421 | 2 out of 3 | 28.57 ± 19.3 | 200 | 365.9 pM |
| c8089 | 2 out of 3 | 26.93 ± 4.8 | 200 | 264 pM |
| c8124 | 2 out of 3 | 57.9 ± 22.3 | 200 | 360.9 pM |
| c8047 | 2 out of 3 | 78.2 ± 19.1 | 200 | 443.7 pM |
| AB001544 | 2 out of 3 | 29.93 ± 7.5 | 200 | 257.4 pM |
| AB-000224 | 1 out of 3 | 29.07 ± 1.8 | 200 | 441.3 pM |
| AB-000315 | 1 out of 3 | 45.07 ± 6.9 | 200 | 62.2 pM |
| mAb B | 1 out of 3 | 89.47 ± 18.8 | 200 | 199.7 pM |
| AB001557 | 1 out of 3 | 36.7 ± 5 | 200 | 1028.2 pM |
| mAb C | 1 out of 3 | 10.78 ± 1 | 200 | 407.9 pM |
| AB001387 | 1 out of 3 | 64.7 ± 2.2 | 200 | 675.8 pM |
| AB-000364 | 1 out of 3 | 73.63 ± 10.9 | 200 | 142.3 pM |
| c8082 | 1 out of 3 | 63.13 ± 9.7 | 200 | 225.5 pM |
| AB-000327 | 0 out of 3 | 11.88 ± 6.8 | 200 | 467.1 pM |
| AB001480 | 0 out of 3 | 60.93 ± 9.8 | 200 | 1175 pM |
| AB001508 | 0 out of 3 | 36.3 ± 11.7 | 200 | 604.4 pM |
| AB001438 | 0 out of 3 | 38.43 ± 3.9 | 200 | 1583.9 pM |
| AB001513 | 0 out of 3 | 51.6 ± 5.5 | 200 | 388.1 pM |
| AB-000349 | 0 out of 3 | 53.3 ± 14.1 | 200 | 1437.5 pM |
| AB-000399 | 0 out of 3 | 43.6 ± 13.1 | 200 | 672.8 pM |
| AB001468 | 0 out of 3 | 99.5 ± 22.3 | 200 | 945.9 pM |
| AB001472 | 0 out of 3 | 82.7 ± 5.2 | 200 | 2107.2 pM |
| AB-000314 | 0 out of 3 | 43.97 ± 6.6 | 200 | 76.9 pM |
| AB001458 | 0 out of 3 | 84.93 ± 10.3 | 200 | 921.1 pM |
| AB001482 | 0 out of 3 | 78.73 ± 15.8 | 200 | 263.3 pM |
| AB001517 | 0 out of 3 | 83.63 ± 9 | 200 | 755.3 pM |
| AB-001395 | 0 out of 3 | 52.9 ± 17.9 | 200 | 1159.6 pM |
| AB-000373 | 0 out of 3 | 57.57 ± 10.8 | 200 | 59.2 pM |
| AB-001428 | 0 out of 3 | 78.63 ± 44.7 | 200 | 1177.2 pM |
| AB-001455 | 0 out of 3 | 30.47 ± 20 | 200 | 1365.9 pM |
| AB-001478 | 0 out of 3 | 74.8 ± 45.7 | 200 | 742.5 pM |
| AB-001533 | 0 out of 3 | 30.6 ± 2.4 | 200 | 248.5 pM |
| c7913 | 0 out of 3 | 57.2 ± 29.7 | 200 | 229.5 pM |
| c7996 | 0 out of 3 | 57.27 ± 31.8 | 200 | 143.3 pM |

TABLE 5
In vivo evaluation of top ten lead antibodies in mouse P.
berghei model at 100 μg dose. All antibodies listed in
the table are half-life extended with M428L/N434S (LS) modification
except for mAb A in one of the studies (noted in the table).

| | In vivo PD (1500 sporozoites | | | PK (serum |
|---|---|---|---|---|
| | i.v challenge) at 100 μg dose | | | concentration) |
| | | % | % | μg/ml at 44 |
| | Complete | parasitemia | parasitemia | hours (100 μg |
| Molecule | protection | at day 5 | at day | dose) |
| name | at day 13 | (Mean ± SD) | 6(Mean ± SD) | (Mean ± SD) |

Study 1

| | | | | |
|---|---|---|---|---|
| mAb A | 1/6 | 0.031 ± | 0.081 ± | 34.70 ± |
| | | 0.013 | 0.119 | 4.32 |
| AB-000356 | 3/6 | 0.05 ± | 0.27 ± | 42.8 ± |
| | | 0.064 | 0.47 | 2.64 |
| AB-000338 | 2/6 | 0.047 ± | 0.17 ± | 27.1 ± |
| | | 0.011 | 0.17 | 4.37 |
| AB001421 | 1/6 | 0.018 ± | 0.28 ± | 16.95 ± |
| | | 0.04 | 0.28 | 4.81 |
| AB-000222 | 0/6 | 0.1 ± | 0.59 ± | 11.53 ± |
| | | 0.043 | 0.35 | 1.94 |
| c8047 | 0/6 | 0.106 ± | 1.07 ± | 31.9 ± |
| | | 0.04 | 0.75 | 6.28 |

Study 2

| | | | | |
|---|---|---|---|---|
| mAb A LS | 0 out of 6 | 0.063 ± | 0.479 ± | 30.02 ± |
| | | 0.04 | 0.317 | 6.69 |
| AB-000394 | 0/6 | 0.063 ± | 0.52 ± | 33.25 ± |
| | | 0.027 | 0.31 | 13.23 |
| AB001544 | 0/6 | 0.045 ± | 0.61 ± | 11.92 ± |
| | | 0.20 | 0.92 | 4.05 |
| c8089 | 0/6 | 0.036 ± | 0.25 ± | 38.11 ± |
| | | 0.03 | 0.28 | 8.09 |
| AB-000334 | 0/6 | 0.085 ± | 0.68 ± | 40.56 ± |
| | | 0.033 | 0.23 | 6.01 |
| c8124 | 0/6 | 0.078 ± | 0.55 ± | 46.45 ± |
| | | 0.052 | 0.38 | 12.48 |

### Example 3 Selection of Amino-Acid Substitutions Necessary to Confer Optimal Fc Effector Function

When incorporating different amino acid substitutions designed to confer increased Fc effector function and half-life into monoclonal antibodies there is a risk that the other attributes of the desired antibody drug profile e.g. target binding affinity, in vivo efficacy or manufacturing potential are compromised.

Therefore, using a tool antibody (mAb A) a range of different amino acid substitution variants were generated. These substitutions were incorporated alongside amino acid substitutions requisite for conferring improved serum half-life. A table showing the Fc variants that were generated is provided (Table 6).

TABLE 6
Table of mAb A Fc variants and other Fc variants generated and
used in the studies. Note, when the molecule's name include
LS it is half-life extended with M428L/N434S modification

| | Abbreviated | |
|---|---|---|
| Modifications to Fc region of | name of | Fc variant tool |
| molecules | mutation set | molecules |
| Parental molecule comparator A | | mAb A |
| M428L/N434S (Half-life extension) | LS | mAb A-LS |
| S239D | SD | mAb A-SD-LS |
| I332E | IE | mAb A-IE-LS |
| S239D/I332E | DE | mAb A-DE-LS |
| S239D/I332E/A330L | DEL | mAb A-DEL-LS |
| G236A/I332E | AE | mAb A-AE-LS |
| S239D/I332E/G236A | DEA | mAb A-DEA-LS |
| G236A/S239D/I332E/A330L | DEAL | mAb A-DEAL-LS |
| H268F/S324T/S239D/I332E | FTDE | mAb A-FTDE-LS |
| H268F/S324T/G236A/I332E | FTAE | mAb A-FTAE-LS |
| S298A/E333A/K334A | AAA | mAb A-AAA-LS |
| F243L/R292P/Y300L/P396L | LPLL | mAb A-LPLL-LS |
| F243L/R292P/Y300L/V305I/P396L | LPLIL | mAb A-LPLIL-LS |
| L235V/F243L/R292P/Y300L/P396L | VLPLL | mAb A-VLPLL-LS |
| F243L/R292P/Y300L | LPL | mAb A-LPL-LS |
| G236A/A330L/I332E | ALE | mAb A-ALE-LS |
| Afucosylated | | mAb A-afucos |
| Half-life extension and afuscosylated | | mAb A-afucos-LS |
| Fc silenced | LAGA | mAb A-Fc silenced |
| Parental molecule | | mAb B |
| Half-life extension M428L/N434S | LS | mAb B-LS |
| S239D/I332E | DE | mAb B-DE-LS |

Protein analytical developability profiling assays were performed including i) protein yield, heparin binding, serum stability, nano DSF and HIC, to assess the potential for efficient manufacturing, ii) human FcRn and CSP target binding to provide confidence that serum half-life and target binding would not be compromised and iii) human FcγR binding by SPR to evaluate potential for improved effector function. Complement C1q binding ELISA studies were also undertaken to evaluate the potential for complement activation by each antibody variant. Beyond in vitro human FcγR binding studies the potential for improved effector function was further assessed on selected variants using an in vitro functional ADNKA assay.

The yield data from 2.0 litre scale Fc variant antibody generation is shown in Table 7. Fc variants mAb-A-DEL-LS and mAb A-DE-LS show surprisingly low total protein yields (30 and 45 mg from 2.0 litre of HEK culture respectively) compared to the other Fc variants that in general had total protein yields greater than 150 mg. As an additional comparison mAb-A generated as an afucosylated molecule had a very low yield from 4 litre of CHO cell culture. For a given antibody therefore, different amino acid substitutions have significantly different impacts on protein yield.

TABLE 7
Fc variants antibody yield data from 2.0 or 4.0 litres of HEK cell culture
and two afucosylated variants from 4 litre of CHO cell culture.

| | | | Scale of | |
|---|---|---|---|---|
| | Conc | Vol | prep | Total yield |
| Molecule name | (mg/ml) | (ml) | (litre) | (mg) |
| mAb A-SD-LS | 3.7 | 46 | 2 | 170 |
| mAb A-IE-LS | 3 | 45 | 2 | 135 |
| mAb A-DE-LS | 5 | 9 | 2 | 45 |
| mAb A-DEL-LS | 3 | 10 | 2 | 30 |
| mAb A-AE-LS | 3 | 50 | 2 | 150 |
| mAb A-DEA-LS | 3 | 50 | 2 | 150 |
| mAb A-DEAL-LS | 3.5 | 45 | 2 | 157 |
| mAb A-FTDE-LS | 4 | 50 | 2 | 200 |
| mAb A-FTAE-LS | 4.5 | 47 | 2 | 211 |
| mAb A-AAA-LS | 5 | 47 | 2 | 235 |
| mAb A-LPLL-LS | 5 | 47 | 2 | 235 |
| mAb A-LPLIL-LS | 4.6 | 48 | 2 | 220 |
| mAb A-VLPLL-LS | 4.5 | 48 | 2 | 216 |
| mAb A-LPL-LS | 4.5 | 48 | 2 | 216 |
| mAb A-ALE-LS | 4.6 | 48 | 2 | 220 |
| mAb A-LS | 4 | 48 | 2 | 192 |
| mAb A-afucos | 2.81 | 24 | 4 | 67.44 |
| mAb-A-afucos-LS | 2.61 | 24 | 4 | 62.64 |

Human FcγR binding data by SPR is shown in Table 8. The affinity data showed a wide range of different FcγR binding profiles with two variants (mAb A-VLPLL-LS and mAb A-LPLL-LS) with significantly different affinities between alleles of the same receptor. Across the variants there was a distinct difference in binding affinities to FcγR IIIa with mAb A-AAA-LS the lowest and mAb A-DEL-LS the highest. The FcγR IIa/IIb binding ratio also differed significantly. Across the FcγR panel mAb-B-DE-LS shows a very similar binding profile to mAb-A-DE-LS indicating that a specific set of amino acid substitutions can confer similarly improved binding affinities to human FcγR's in different antibodies.

TABLE 8
Table showing Fc variants SPR affinity data using human FcγR's.

| | Human FcγR's binding affinity KD (nM) | | | | | |
|---|---|---|---|---|---|---|
| | | | hFcγRIIa | | hFcγRIIIa | hFcγRIIIa |
| Molecule name | hFcγRI | hFcγRIIa (H131) | (R131) | hFcγRIIb | (V158) | (F158) |
| mAb A-SD-LS | 14.1 | 259 | 186 | 352 | 26.7 | 117 |
| mAb A-IE-LS | 16 | 230 | 297 | 459 | 31.5 | 113 |
| mAb A-DE-LS | 18.8 | 136 | 110 | 128 | 9.65 | 21.5 |
| (batch 1) | | | | | | |
| mAb A-LS-DE | 11 | 126 | 101 | 113 | 9.12 | 23.7 |
| (batch 2) | | | | | | |
| mAb A-DEL-LS | 16.6 | 287 | 230 | 246 | 7.89 | 12.9 |
| mAb A-AE-LS | 26.3 | 43.7 | 86.2 | 420 | 49.7 | 125 |
| mAb A-DEA-LS | 20.6 | 32.1 | 33.2 | 140 | 11.2 | 21.7 |
| mAb A-DEAL-LS | 15.9 | 67.9 | 65.6 | 246 | 9.28 | 14.7 |
| mAb A-FTDE-LS | 15.6 | 76.2 | 75.5 | 90.9 | 9.49 | 26.1 |
| mAb A-FTAE-LS | 20.9 | 23.4 | 82 | 365 | 50.1 | 120 |
| mAb A-AAA-LS | 22.9 | 1140 | 997 | 1130 | 58.7 | 181 |
| mAb A-LPLL-LS | 23.6 | 136 | 344 | 558 | 19.3 | 75.3 |
| mAb A-LPLIL-LS | 23.8 | 130 | 327 | 517 | 16.4 | 60.8 |
| mAb A-VLPLL-LS | 31.6 | 304 | 1200 | 1130 | 32.4 | 102 |
| mAb A-LPL-LS | 22.3 | 281 | 703 | 933 | 32.1 | 163 |
| mAb A-ALE-LS | 16.8 | 103 | 199 | 792 | 47.6 | 93.3 |
| mAb A-LS | 20 | 252 | 356 | 620 | 148 | 651 |
| mAb A | 21.1 | 380 | 565 | 904 | 155 | 685 |
| mAb A-Fc-silenced | NB | NB | weak/NSB | weak/NSB | NB | NB |
| mAb B | 32.6 | 426 | 616 | 1430 | 192 | 729 |
| mAb B-LS | 29.1 | 344 | 478 | 1170 | 156 | 586 |
| mAb B-DE-LS | 15.3 | 150 | 121 | 157 | 12.3 | 30.3 |
| mAb A-afucos | 22.7 | 423 | 442 | 724 | 14.9 | 55.3 |
| mAb A-afucos-LS | 22.2 | 375 | 394 | 655 | 14.6 | 50.4 |
| hIgG1 WT control | 19.2 | 310 | 380 | 1150 | 74.2 | 195 |
| hIgG1 Fc silenced | NB | NB | weak/NSB | weak/NSB | NB | NB |
| control | | | | | | |

NB = no binding,
NSB = no significant binding

Nano-DSF data (Table 9) shows a clear melting profile trend with all DEA, DEL and DEAL containing variants having a Tm1 melting inflection point at 50° C. or below followed by a group of variants with Tm1 in the 51-60° C. range, and three variants with Tm1 at 60-65° C., The mAb-A (Wildtype) molecule has Tm1 at 69.45° C. Tm1 of mAb-B-DE-LS is 51.39° C. and compares very closely to the measured Tm1 of mAb-A-DE-LS (51.13° C.) showing that a specific set of amino acid substitutions can have similar impact across different antibodies. Tagg, the onset of aggregation is fairly consistent across the mAb-A variant molecules at 72/73° C. and likely reflects melting of the Fab in each case whilst Tagg for mAb-B is several

TABLE 9
nano-DSF showing melt profiles of Fc variants panel.

| Antibody name | Tm1 ° C. | Tm2 ° C. | Tm3 ° C. | Tagg ° C. |
|---|---|---|---|---|
| mAb A-DEAL-LS | 48.96 | 70.40 | 78.57 | 72.74 |
| mAb A-DEL-LS | 49.63 | 70.50 | 78.81 | 73.17 |
| mAb A-DEA-LS | 50.34 | 69.89 | 78.49 | 73.02 |
| mAb A-DE-LS | 51.13 | 70.29 | 78.56 | 73.02 |
| mAb A-FTDE-LS | 53.31 | 70.29 | 78.79 | 73.01 |
| mAb A-ALE-LS | 57.64 | 71.51 | 78.96 | 72.49 |
| mAb A-AE-LS | 58.95 | 69.89 | 78.28 | 72.87 |
| mAb A-IE-LS | 59.30 | 70.09 | 78.74 | 72.93 |
| mAb A-FTAE-LS | 60.52 | 71.10 | 78.96 | 72.27 |
| mAb A-AAA-LS | 64.09 | 71.71 | 79.04 | 72.16 |
| mAb A-SD-LS | 65.06 | 78.50 | n/a | 72.61 |
| mAb A-afucos-LS | 68.92 | 78.76 | n/a | 72.34 |
| mAb A-LS (batch 1) | 68.96 | 78.96 | n/a | 72.47 |
| mAb A-LS (batch 2) | 69.42 | 79.07 | n/a | 73.02 |
| mAb A-Fc-silenced | 69.35 | 82.95 | n/a | 73.24 |
| mAb A | 69.45 | 82.92 | n/a | 72.95 |
| mAb A-LPLIL-LS | 69.60 | n/a | n/a | 71.98 |
| mAb A-afucos | 69.66 | 82.92 | n/a | 73.16 |
| mAb A-LPLL-LS | 69.77 | n/a | n/a | 72.10 |
| mAb A-VLPLL-LS | 69.92 | n/a | n/a | 72.16 |
| mAb A-LPL-LS | 70.18 | 79.47 | n/a | 72.55 |
| mAb B | 67.04 | 82.31 | 89.43 | 69.47 |
| mAb B-LS | 66.91 | 78.36 | n/a | 69.15 |
| mAb B-DE-LS | 51.39 | 64.56 | 78.66 | 68.22 |

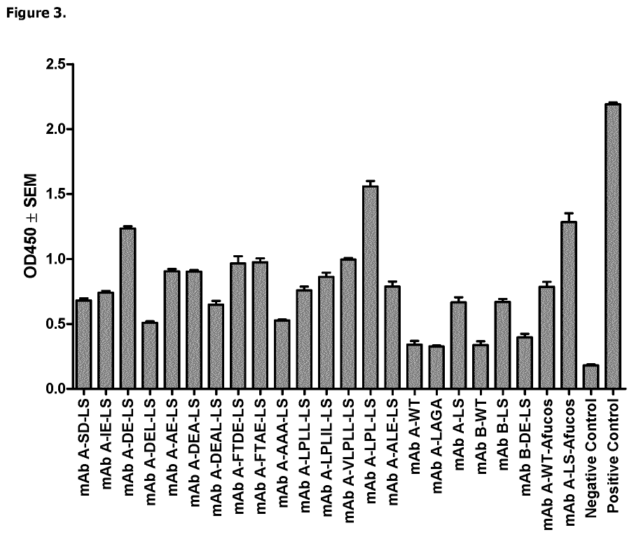

The literature demonstrates that heparin binding has a strong correlation to poor antibody pharmacokinetics in vivo (Datta-Mannan et al, 2015 Mabs, 7(3):483-493) and the positive control antibody used in the heparin-binding assay is known to have poor pharmacokinetics. The heparin binding ELISA data (FIG. 3) shows that all Fc variants exhibit heparin binding below the positive control antibody but above the negative control. Fc variants mAb A-DE-LS, mAb A-LS-afucose and mAb A-LPL-LS show the highest mean heparin binding values in this assay.

Complement C1q ELISA assay data (Table 10) shows a highly variable binding profile across the different Fc variants. Variant mAb A-IE, mAb A-AE and mAb-A SD have high C1q binding whilst DE containing variants, in particular mAb A-DEL-LS and mAb A-DEAL-LS, and also mAb A-ALE-LS generally have reduced C1q binding at or close to background levels. Moreover, different CSP antibodies have different C1q binding profiles example mAb A-LS, and mAb B-LS.

TABLE 10
Complement C1q ELISA binding data on the panel of Fc variants.

| | | | | C1q |
|---|---|---|---|---|
| | | EC50 C1q | | binding |
| | C1q (nM) | binding | % of WT at | High/ |
| | at 20% | to mAb | maximum | Medium/ |
| Antibody name | Threshold | (nM C1q) | C1q (21 nM) | Low |
| CTRL-WT | 0.4114569 | 4.05 | 101.41 | High |
| CTRL-WT | 0.2994974 | 3.159 | 101.40 | High |
| CTRL-WT | 0.38635 | 3.351 | 106.62 | High |
| CTRL-WT | 0.3443095 | 4.853 | 108.12 | High |
| CTRL-Fc-silenced | 16.0861 | 8.546 | 20.64 | Low |
| CTRL-Fc-silenced | 17.78779 | 17.25 | 23.32 | Low |
| CTRL-Fc-silenced | 20.4057 | 1753 | 21.91 | Low |
| CTRL-Fc-silenced | 18.91261 | ~231.6 | 23.40 | Low |
| mAb A-SD-LS | 0.3481189 | 1.801 | 104.44 | High |
| mAb A-IE-LS | 0.2670709 | 1.035 | 105.85 | High |
| mAb A-DE-LS | 1.698536 | — | 88.98 | Medium |
| (batch 1) | | | | |
| mAb A-DE-LS | 1.313326 | — | 95.71 | High |
| (batch 2) | | | | |
| mAb A-DEL-LS | 6.605118 | — | 22.84 | Low |
| mAb A-AE-LS | 0.3809641 | 6.576 | 105.21 | High |
| mAb A-DEA-LS | 3.880605 | — | 62.10 | Medium |
| mAb A-DEAL-LS | — | — | 11.88 | — |
| mAb A-FTDE-LS | 0.1797744 | 0.4494 | 111.03 | High |
| mAb A-FTAE-LS | 0.1389826 | 0.3218 | 113.60 | High |
| mAb A-AAA-LS | 0.1705267 | 0.4475 | 113.48 | High |
| mAb A-LPLL-LS | 0.1325286 | 0.2897 | 113.02 | High |
| mAb A-LPLIL-LS | 0.1051902 | 0.3174 | 113.81 | High |
| mAb A-VLPLL-LS | 0.178035 | 0.5175 | 113.90 | High |
| mAb A-LPL-LS | 0.1684772 | 0.375 | 113.74 | High |
| mAb A-ALE-LS | — | — | 14.19 | — |
| mAb A-LS | 0.1866467 | 0.5126 | 110.13 | High |
| mAb A | 0.1680205 | 0.519 | 114.78 | High |
| mAb A-Fc-silenced | 5.067783 | — | 55.05 | Medium |
| mAb B | 0.2543223 | 1.843 | 104.36 | High |
| mAb B-LS | 0.3148159 | 3.294 | 102.57 | High |
| mAb B-DE-LS | 9.287024 | — | 40.80 | Low- |
| | | | | medium |
| mAb A-LS | 0.218153 | 0.6764 | 113.84 | High |
| mAb A-afucos | 0.1636228 | 0.5272 | 117.98 | High |
| mAb A-afucos-LS | 0.1765699 | 0.5387 | 117.26 | High |

Human FcRn binding by SPR (Table 11) shows that the introduction of a range of different amino acid substitutions to improve effector function does not alter binding to human FcRn and so on its own would not be anticipated to significantly impact pharmacokinetics in vivo.

TABLE 11
Fc enhancing amino acid substitutions do not alter human FcRn binding
affinities (NB = non binding, WB = weak binding).

| | Temp | KD (nM) | KD (nM) |
|---|---|---|---|
| Antibody | (° C.) | pH 6 | pH 7.4 |
| mAb A-Fc-silenced | 25 | 864 | NB |
| mAb A | 25 | 896 | NB |
| mAb A-afucos | 25 | 949 | NB |
| mAb B | 25 | 926 | NB |
| mAb B-LS | 25 | 90.1 | WB |
| mAb B-DE-LS | 25 | 117 | WB |
| mAb A-LS | 25 | 104 | WB |
| mAb A-afucos-LS | 25 | 97.5 | NB |
| mAb AmAb A-SD-LS | 25 | 86.5 | WB |
| mAb A-IE-LS | 25 | 89.7 | WB |
| mAb A-DE-LS (batch 1) | 25 | 94.3 | WB |
| mAb A-DE-LS (batch 2) | 25 | 82.5 | WB |
| mAb A-DEL-LS | 25 | 90.5 | WB |
| mAb A-AE-LS | 25 | 88.7 | WB |
| mAb A-DEA-LS | 25 | 84 | WB |
| mAb A-DEAL-LS | 25 | 85 | WB |
| mAb A-FTDE-LS | 25 | 88 | WB |
| mAb A-FTAE-LS | 25 | 90.3 | WB |
| mAb A-AAA-LS | 25 | 91.3 | WB |
| mAb A-LPLL-LS | 25 | 82.2 | WB |
| mAb A-LPLIL-LS | 25 | 78.8 | WB |
| mAb A-VLPLL-LS | 25 | 83.1 | WB |
| mAb A-LPL-LS | 25 | 98.1 | WB |
| mAb A-ALE-LS | 25 | 85.7 | WB |
| mAb A-LS | 25 | 85.8 | WB |

Target CSP binding affinity is unchanged by the introduction of Fc substitutions (Table 12) and similarly, hydrophobic interaction chromatography (HIC) did not show any significant differences among the Fc variants (Table 13).

The in vitro stability, following incubation in human serum, of a panel of anti-CSP monoclonal antibodies was determined over an 8 week period. Each antibody was spiked into a pooled stock of human serum (n=12 donors) to a concentration of 120 μg/ml. Seven individual samples were prepared from each stock and in each case all bar one sample (designated TO) were incubated at 37° C. for a predetermined period of time (1, 2, 3, 4, 5 or 8 weeks). TO samples were immediately frozen on dry ice then stored at −80° C. until all subsequent samples had been collected and frozen in a similar manner.

Using CSP coated plates for capture and a sulfotagged anti human IgG1 Fc mAb for detection, an MSD (Meso Scale Discovery) immunoassay was used to quantify functional antibody remaining in each set of samples following incubation. Standard curves were prepared (using unstressed, sample matched material) against which test samples were interpolated and a concentration determined. Recovery from each sample was then expressed as a percentage of its initial T0 sample.

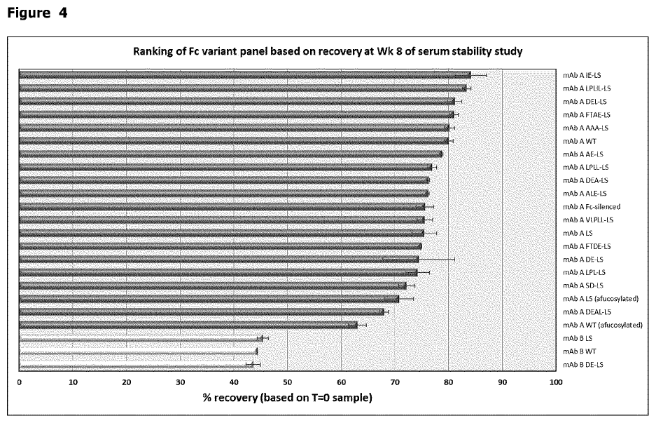

Whilst human serum stability data shows no difference between Fc variants of the same molecule (FIG. 4) there are detectable differences between different antibodies for example mAb A molecules and mAb B molecules.

TABLE 12
Fc enhancing amino acid substitutions do
not alter CSP target binding affinity

| Antibody | Ka (1/Ms) | Kd (1/s) | KD (M) |
|---|---|---|---|
| mAb A-SD-LS | 1.11E+06 | 2.02E−04 | 1.82E−10 |
| mAb A-IE-LS | 2.34E+06 | 7.02E−04 | 3.01E−10 |
| mAb A-DE-LS | 1.77E+06 | 4.85E−04 | 2.73E−10 |
| mAb A-DEL-LS | 2.80E+06 | 7.88E−04 | 2.81E−10 |
| mAb A-AE-LS | 1.78E+06 | 5.03E−04 | 2.82E−10 |
| mAb A-DEA-LS | 1.82E+06 | 6.80E−04 | 3.73E−10 |
| mAb A-DEAL-LS | 1.49E+06 | 3.53E−04 | 2.36E−10 |
| mAb A-FTDE-LS | 2.00E+06 | 4.05E−04 | 2.03E−10 |
| mAb A-FTAE-LS | 1.11E+06 | 2.21E−04 | 2.00E−10 |
| mAb A-AAA-LS | 1.38E+06 | 2.50E−04 | 1.81E−10 |
| mAb A-LPLL-LS | 1.99E+06 | 4.15E−04 | 2.09E−10 |
| mAb A-LPLIL-LS | 2.37E+06 | 5.63E−04 | 2.38E−10 |
| mAb A-VLPLL-LS | 1.47E+06 | 3.18E−04 | 2.16E−10 |
| mAb A-LPL-LS | 1.73E+06 | 5.67E−04 | 3.28E−10 |
| mAb A-ALE-LS | 1.75E+06 | 3.56E−04 | 2.03E−10 |
| mAb A-LS | 1.22E+06 | 2.52E−04 | 2.07E−10 |
| mAb A-afucos | 1.93E+06 | 1.96E−04 | 1.02E−10 |
| mAb A-afucos-LS | 2.14E+06 | 3.88E−04 | 1.81E−10 |

TABLE 13
Fc enhancing amino acid substitutions
do not alter HIC retention time

| | | Peak | Main | Peak Width |
|---|---|---|---|---|
| Name | RT | Area | Area % | 50% |
| hIgG1 control (replicate 1) | 1.239 | 1965.01 | 100 | 0.075 |
| hIgG1 control (replicate 2) | 1.251 | 1893.93 | 100 | 0.074 |
| mAb A-SD-LS | 1.722 | 2290.74 | 100 | 0.063 |
| mAb A-IE-LS | 1.718 | 2088.56 | 100 | 0.063 |
| mAb A-DE-LS | 1.724 | 2003.21 | 93.33 | 0.063 |
| mAb A-DEL-LS | 1.73 | 2319 | 94.82 | 0.061 |
| mAb A-AE-LS | 1.728 | 2197.18 | 100 | 0.063 |
| mAb A-DEA-LS | 1.736 | 1895 | 100 | 0.063 |
| mAb A-DEAL-LS | 1.741 | 2274.36 | 100 | 0.062 |
| mAb A-FTDE-LS | 1.734 | 2227.51 | 100 | 0.062 |
| mAb A-FTAE-LS | 1.737 | 2499.15 | 100 | 0.062 |
| mAb A-AAA-LS | 1.731 | 2435.51 | 100 | 0.062 |
| mAb A-LPLL-LS | 1.744 | 2379.66 | 100 | 0.063 |
| mAb A-LPLIL-LS | 1.744 | 2138.34 | 100 | 0.064 |
| mAb A-VLPLL-LS | 1.746 | 2093.79 | 100 | 0.062 |
| mAb A-LPL-LS | 1.73 | 2381.57 | 100 | 0.062 |
| mAb A-ALE-LS | 1.735 | 2492.77 | 100 | 0.061 |
| mAb A-LS (batch 1) | 1.716 | 2963.19 | 100 | 0.065 |
| mAb A-LS (batch 2) | 1.718 | 2710.42 | 100 | 0.06 |
| mAb A | 1.687 | 2866.02 | 100 | 0.059 |
| mAb A-Fc-silenced | 1.694 | 2593.69 | 100 | 0.058 |
| mAb A-DE-LS | 1.725 | 2724.26 | 100 | 0.058 |
| mAb B | 1.17 | 2701.41 | 100 | 0.065 |
| mAb B-LS | 1.218 | 2711.34 | 100 | 0.064 |
| mAb B-DE-LS | 1.216 | 2758.31 | 100 | 0.064 |
| mAb A-afucos | 1.693 | 2424.78 | 100 | 0.064 |
| mAb A-afucos-LS | 1.723 | 2493.09 | 100 | 0.064 |

Nine Fc variants were selected on the basis of the above data as having favourable physical properties and predicted enhanced Fc function (based on FcγR binding affinity). These Fc variants were further triaged down to three using data generated using an in vitro human antibody-dependent NK cell activation (ADNKA) assay (Table 14) in which recombinant CSP is coated onto a microtitre plate, and incubated with antibodies followed by NK cells from human donors. NK activation markers are then measured by flow cytometry. These studies show that all Fc modifications tested enhance ADNKA in the presence of recombinant CSP, in particular those containing the ‘DE’ mutations and the ‘LPLIL’ variant. DEA, FTDE and ALE were selected from this panel to ensure the variants taken forward for further evaluation represented those that would enhance the full range of Fc-dependent mechanisms based on published examples and from the binding affinities to human FcγRs shown in Table 8. As a result of this work and to address the low Tm1 temperature associated with the DEA variant, T250V/A287F (V/F) mutations were added to the DEA variant for future studies.

TABLE 14
In vitro evaluation of Fc enhanced antibodies in an antibody-dependent natural killer cell
activation (ADNKA) assay

| pEC50 | DE | AE | DEA | DEAL | FTDE | FTAE | AAA | LPLIL | ALE | LS | WT | Fc silenced |
|---|---|---|---|---|---|---|---|---|---|---|---|---|
| Donor 1 | 10.83 | 10.72 | 11.05 | 10.93 | 10.89 | 10.61 | 10.42 | 10.46 | 10.48 | 10.18 | 10.28 | <7.17 |
| Donor 2 | 10.70 | 10.41 | 10.56 | 10.70 | 10.66 | 10.06 | 9.88 | 10.40 | 9.86 | 9.45 | 9.85 | <7.17 |
| Donor 3 | 9.10 | 9.29 | 9.32 | 9.25 | 11.06 | 10.42 | 10.39 | 10.98 | 10.54 | 9.63 | 9.90 | <7.17 |
| Donor 4 | 11.24 | 10.65 | 11.16 | 11.09 | 10.95 | 10.52 | 10.51 | 10.93 | 10.65 | 10.25 | 10.16 | <7.17 |
| Donor 5 | 10.98 | 10.42 | 10.86 | 10.66 | 11.05 | 10.32 | 10.40 | 10.83 | 10.42 | 9.89 | 10.06 | <7.17 |
| Donor 6 | 10.56 | 10.17 | 10.65 | 10.54 | 10.81 | 10.28 | 10.25 | 10.68 | 10.41 | 9.84 | 9.99 | <7.17 |
| Donor 7 | 10.28 | 9.70 | 10.49 | 10.43 | 10.22 | 10.00 | 10.54 | 10.51 | 10.53 | 9.72 | 9.60 | <7.17 |

### Example 4 Incorporation of Amino Acid Substitutions Conferring Optimal Fc Effector Function into the Most Efficacious Lead Antibodies

The amino acid substitutions selected from Example 3 were each incorporated into the sequence of the most efficacious antibodies, mAb 356 and mAb 338 identified in in vivo studies in Example 2. Functional in vitro assays were undertaken in order to confirm that improved effector function could be transferred to these most potent molecules identified from in vivo studies. The molecules generated were 338-ALE-LS, 356-ALE-LS and 356-DEA-FV-LS. FTDE-LS variants were not evaluated further. In an ADCP assay measuring phagocytosis of recombinant CSP-coated beads by the monocyte cell line THP-1, all of the molecules tested were more potent than the mAb A comparator (including the mAb A variants with the DE and FTAE Fc-modifications). Both of the 356 variants, 356-ALE-LS and 356-DEA-FV-LS were more potent than any of the other molecules tested with EC50s of <1 pM (Table 15). In an ADNKA assay, all of the ALE and DEA-FV containing molecules elicited enhanced ADNKA compared with their LS only versions (Table 16).

TABLE 15a
In vitro evaluation of lead antibodies in an antibody-dependent
cellular phagocytosis (ADCP) assay - Mean from 2 replicates

| | GeoMean | Mean EC50 | |
|---|---|---|---|
| .mAb | EC50 (M) | (pM) | Mean pEC50 |
| mAb A FTAE-LS | 2.19E−11 | 21.9 | 10.66 |
| mAb A LS | 2.25E−11 | 22.5 | 10.65 |
| mAb A DE-LS | 5.80E−12 | 5.8 | 11.24 |
| AB-000338 LS | 9.19E−12 | 9.2 | 11.04 |
| AB-000356 LS | 9.27E−12 | 9.3 | 11.03 |
| AB-000356 ALE LS | 5.03E−13 | 0.5 | 12.30 |
| AB-000338 ALE LS | 1.18E−11 | 11.8 | 10.93 |
| AB-000356 DEA FV LS | 7.88E−13 | 0.8 | 12.10 |
| AB-000338 DEA FV LS | 7.21E−12 | 7.2 | 11.14 |

TABLE 15b
In vitro evaluation of lead antibodies repeated
in (ADCP) assay with 4 replicates per mean value

| | GeoMean | Mean EC50 | Mean | |
|---|---|---|---|---|
| mAb | EC50 (M) | (pM) | pEC50 | n |
| AB-000338 LS | 1.08E−11 | 10.8 | 10.97 | 4 |
| AB-000356 ALE LS | 6.45E−13 | 0.6 | 12.19 | 4 |
| AB-000338 ALE LS | 6.55E−12 | 6.6 | 11.18 | 4 |
| AB-000356 DEA FV LS | 1.35E−12 | 1.4 | 11.87 | 4 |

TABLE 16
In vitro evaluation of lead antibodies in an antibody-
dependent natural killer cell activation assay (ADNKA)
Geo Mean EC50 (ug/ml)

| mAb | Donor 1 | Donor 2 | Donor 3 | Donor 4 |
|---|---|---|---|---|
| 317 LS | 0.0567 | 0.231 | 0.1513 | 0.0709 |
| CIS43 LS | 0.0277 | 0.1426 | 0.1044 | 0.0643 |
| L9 LS | 0.038 | 0.5143 | 0.0887 | 0.0286 |
| 317 DE-LS | 0.0027 | 0.0022 | 0.0276 | 0.0111 |
| AB-000356 ALE LS | 0.0052 | 0.0035 | 0.1886 | 0.0104 |
| AB-000338 LS | 0.0301 | 0.2376 | 0.1714 | 0.0887 |
| AB-000356 LS | 0.0704 | 1.5013 | 1.3069 | 0.1094 |
| AB-000338 ALE LS | 0.0035 | 0.0037 | 0.0802 | 0.0214 |
| AB-000356 DEA FV LS | 0.0038 | 0.0019 | 0.0165 | 0.0054 |
| AB-000338 DEA FV LS | 0.0087 | 0.0057 | 0.0272 | 0.0067 |

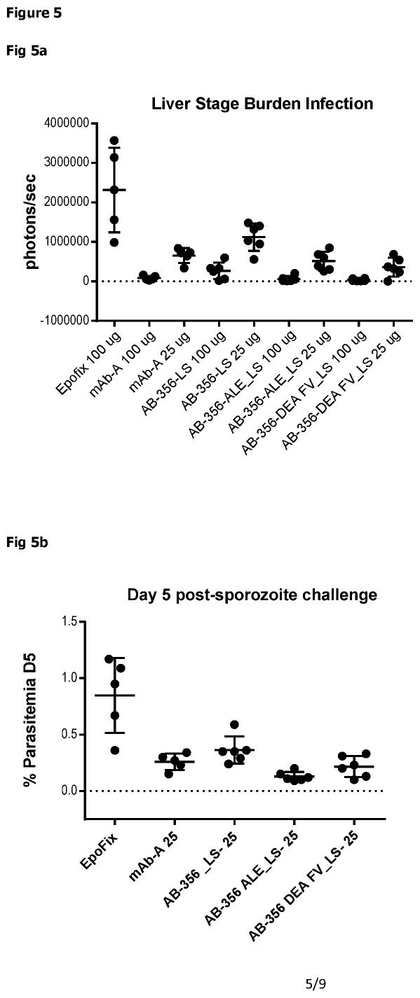

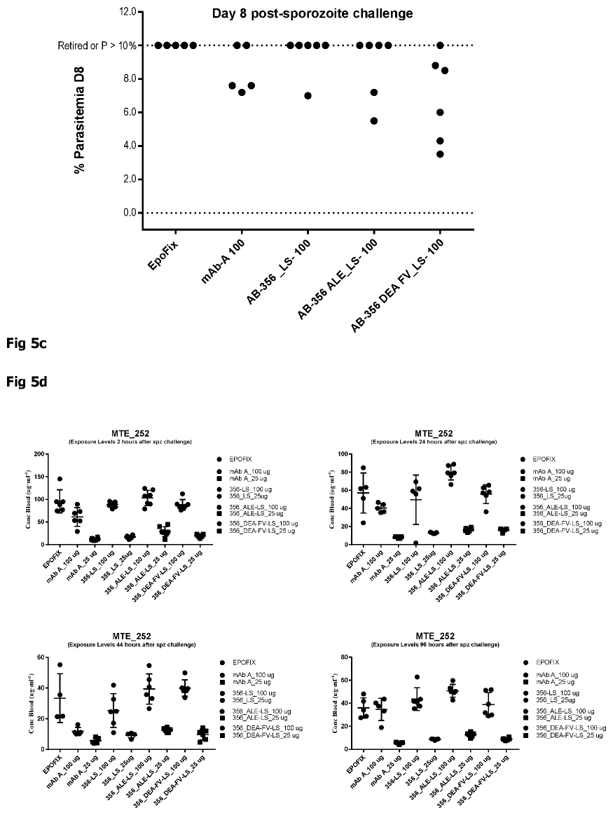

To evaluate the in vivo impact of improved effector function antibodies 356-ALE-LS and 356-DEA-FV-LS were compared to mAb 356-LS in the P. berghei mouse model. The study protocol is shown in table 17 and the data shown in FIG. 5. The serum concentrations of each mAb at 2 hours 24 hours, 44 and 96 hours post dosing are also shown. EpoFix (100 μg dose), and mAb A-LS (25 μg and 100 μg doses) were used as positive and negative controls of infection.

TABLE 17

| | | | Antibody | | |
|---|---|---|---|---|---|
| Mice | | | administered | | PK |
| per | | Dose i.v | prior to spz | Spz (i.v) | timepoints |
| group | Antibody name | (μg) | challenge | challenge | (hours) |
| 5 + 1 | Epo-FIX CTRL | 100 | 24 h | 1500 | 2, 24, 44, |
| | | | | | 96, 168, 336 |
| 5 + 1 | mAb-A-LS | 100 | 24 h | 1500 | 2, 24, 44, |
| | | | | | 96, 168, 336 |
| 5 + 1 | mAb-A-LS | 25 | 24 h | 1500 | 2, 24, 44, |
| | | | | | 96, 168, 336 |
| 6 + 1 | 356 -LS | 100 | 24 h | 1500 | 2, 24, 44, |
| | | | | | 96, 168, 336 |
| 6 + 1 | 356-LS | 25 | 24 h | 1500 | 2, 24, 44, |
| | | | | | 96, 168, 336 |
| 6 + 1 | 356 -ALE-LS | 100 | 24 h | 1500 | 2, 24, 44, |
| | | | | | 96, 168, 336 |
| 6 + 1 | 356 -ALE-LS | 25 | 24 h | 1500 | 2, 24, 44, |
| | | | | | 96, 168, 336 |
| 6 + 1 | 356-DEA-FV- | 100 | 24 h | 1500 | 2, 24, 44, |
| | LS | | | | 96, 168, 336 |
| 6 + 1 | 356 -DEA- | 25 | 24 h | 1500 | 2, 24, 44, |
| | FV-LS | | | | 96, 168, 336 |

A statistical analysis of the PK/PD relationship between 356-ALE-LS with 356-LS showed that the effect of Fc enhancement is small with a probability that Fc-enhanced mAb 356-ALE-LS is at least 1 fold more potent than 356-LS of near to or, above 90%. Specific probabilities were 87.60% on day 2, 99.96% day 3 and, 94.96% on day 5. These probabilities fall to 21.06% (day 2), 61.76% (day 3) and 5.55% (day 5) for a three-fold improvement in protective efficacy compared the 356-LS molecule.

This very narrow difference in efficacy between 356-ALE-LS and 356-LS observed in the P. berghei model is not consistent with the much wider differences seen in the in vitro functional Fc assays described in Example 4. Given the known differences between human and mouse FcγR activity and cell distribution there is a possibility that the mouse C57BL/6 mice background does not reflect a level of Fc enhanced antibody activity in keeping with human mAbs as such further in vivo studies will make use of mice that are transgenic for human FcγR's.

### Example 5—Safety Analysis of Lead Antibodies

Various assays and tests on the four lead antibodies were carried out to ensure as far as possible (in an in vitro) setting that the antibodies would be safe to develop in respect of for example, immunogenicity, tissue cross reactivity etc. Brief results are summarized below:—
i) Immunogenicity testing on the 4 leads, 356-ALE-LS, 356-DEA-FV-LS, 338-LS and 338-ALE-LS, was performed using human peripheral blood mononuclear cells from 20 different donors as the source of CD4+ T cells and dendritic cells, and CD4+ T cell proliferation and IFNγ production as readout. The results provided a general assessment between the four molecules 356-ALE-LS induced 2 positive responses out of 17 donors (12% response rate), 356-DEA-FV-LS induced 5 positive responses out of 17 donors (29% response rate), and both, 338-ALE-LS and 338-LS induced 3 positive responses out of 16 donors (19% response rate). Based on assay qualification results and acccumulative data from in-house immunogencity risk esting, these results suggest that these mAbs are likely to exhibit comparable potential to induce immune responses.
ii) Non-GLP human Tissue Cross Reactivity (TCR) was performed with the four lead mAbs in testis, kidney, skin, tonsil and eye tissue and showed no positive staining that was unspecific binding of the mAbs, in any of the five human tissues.
iii) Human off-target binding assessment was performed using cell microarray technology (Retrogenix), in which a panel of full-length plasma membrane proteins, plasma membrane tethered-secreted proteins and a set of heterodimers, (more than 6400 human proteins), was tested for binding to the 4 mAb leads at 20 μg/ml of mAb. Specific interaction was observed between SPOCK1 protein and two of the mAbs, 356-ALE-LS and 356-DEA-FV-LS. mAbs 338-LS and 338-ALE-LS showed a specific interaction to CNTNAP2. To further investigate those results, further SPR binding data was generated using recombinant SPOCK1 and CNTNAP2 proteins and the 4 mAbs. These SPR results confirmed that none of the antibodies showed binding above the non-specific binding to the surface of 300 nM of protein.
iv) SCISSOR (subcutaneous injection site simulator) is an in vitro assay designed to test molecules for bioavailability subsequent to subcutaneous injection. The predicted value for clinical bioavailability from SCISSOR analysis of GSK4425689A was 51.90% (NCR2022N506638_00). For context, this value compares to the bioavailability of a small set of approved comparator mAbs of between 52 and 80% (Zhao L et al. 2013).

### Example 6 In Vitro and In Vivo Assessment of Fc Enhanced mAb's in Mice Transgenic for Human Fcγ Receptors

Whilst there are examples of the use of mouse models to show that Fc enhanced mAbs have improved efficacy Hiatt et al, 2014, PNAS 111; 5992-5997; DiLillo et al, 2014, Nat Med 20(2) 143-151). the known differences in effector cell distribution between human and mouse Fcγ receptors makes the translational value of wild-type mouse models uncertain. Therefore, mice that are transgenic for human Fcγ receptors have previously been developed (Smith et al, 2012, Proc Natl Acad Sci USA., 109(16): 6181-6186) and, used in order to better predict the translational value of mAbs in disease (Casey et al, 2018, Leukemia, 32(2): 547-549.

Our previous studies have shown a wide difference between the >20 fold improvement in the in vitro activity of Fc enhanced molecules compared to the very small in vivo improvement in C57BL/6 mice so the possibility that the C57BL/6 mouse P. berghei model underrepresents the translational efficacy of Fc enhanced mAbs will be explored using mice that are transgenic for human Fcγ receptors.

In vitro studies aimed at validating the transgenic mice will be undertaken, specifically intending to show that NK cells isolated from the transgenic mice but not NK cells isolated from C57BL/6 mice respond to activation in an assay using antibodies bound to immobilised CSP. These data will provide confidence that Transgenic mice can better reflect the true translational value of mAbs that are designed to enhance NK cells (and other effector cells) against malaria sporozoites and moreover explain why C57BL/6 mice respond poorly to such Fc enhanced mAbs.

Following the in vitro work, in vivo studies using the transgenic mice were undertaken to demonstrate that Fc enhanced mAbs show a greater margin of efficacy compared to our prior studies in P. berghei infected C57BL/6 mice that are shown in FIG. 5.

### Example 7 In Vitro and In Vivo Assessment of Fc Enhanced mAb's in Mice Transgenic for Human Fcγ Receptors

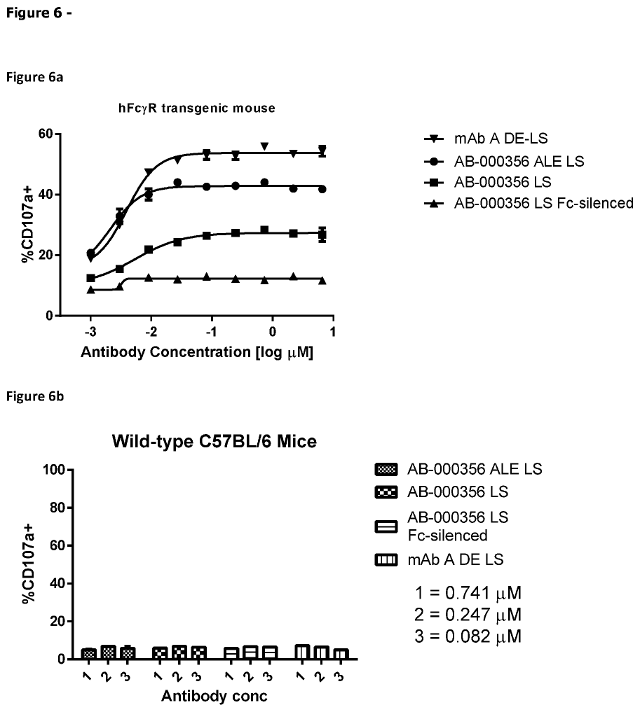

In vitro studies used to evaluate the potential for activation of NK cells isolated from both wild-type C57BL/6 mice and mice transgenic for human Fcγ receptors comprised recombinant CSP coated onto a microtitre plate and incubated with anti-CSP mAbs followed by addition of NKs isolated from mouse splenocytes. The activation marker CD107a was measured by flow cytometry. The difference in response between mAbs AB-000356 LS and AB-000356 ALE LS in human FcγR transgenic mice show that Fc-enhancing substitutions can enhance NK cell activation (FIG. 6a), whilst studies using the wild-type C57/Bl6 mice (FIG. 6b) show no NK activation signal over baseline. These in vitro studies may in part explain why C57BL/6 mice respond poorly to Fc enhanced mAbs.

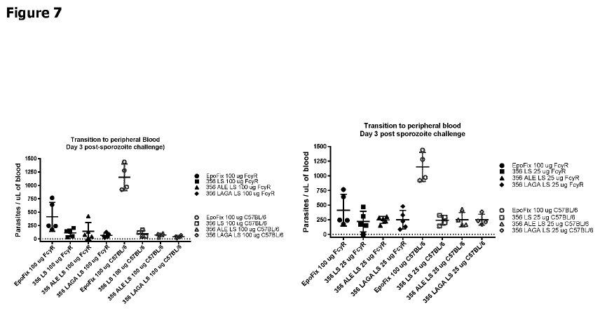

In vivo studies were then undertaken in the mice transgenic for the human Fcγ receptors. Briefly, two antibody doses (25 μg and 100 μg per mouse) were administered by the i.v route to both transgenic mice (n=5 per group) and WT C57BL/6 mice (n=4 per group) 24 hours before challenge with 1500 sporozoites. Liver infection was evaluated at 44 hours and blood stage infection by PCR at day 3 and FACS at 5, 6, 8 days. The antibody variants used in the study were 356-LS, 356-LAGA and 356 ALE-LS. A negative control antibody (EpoFix) was also included in the study. Blood parasitemia levels at day 3 after sporozoite challenge are are shown in FIG. 7 and the summary data is shown in Table 18 and 19. No differences in the level of protection were evident between the different antibodies with all antibodies showing a similar level of protection compared to the negative control antibody. The pharmacokinetic data indicates a broadly similar concentration of antibody in blood between molecules.

TABLE 18
Summary of study data: 25 ug dose groups

| | | | | | | | | Antibody |
|---|---|---|---|---|---|---|---|---|
| | | | | | | | | Serum |
| | | | | | | | | Concentration |
| | | | | qPCR | FACS | FACS | FACS | (mg/ml) |
| Genetic | | Treatment | Total Flux | Parasites/ | % | % | % | 24 hour |
| Background | Mouse | Dose | p/s | ml | Parasitemia | Parasitemia | Parasitemia | after |
| Mice | ID | (25 μg) | 44 hours | Day 3 | Day 5 | Day 6 | Day 8 | IV admin |
| huFcγR | N1♂ | 356 LS | 34820 | 151.48 | 0.60 | 4.22 | Retired | 6.77 |
| Transgenic | N2♂ | | 137200 | 168.07 | 0.73 | 4.78 | Retired | 6.84 |
| Mice | N3♂ | | 1535000 | Retired | Retired | Retired | Retired | 4.13 |
| | N4♀ | | 2449000 | 468.00 | 0.78 | 4.78 | Retired | 10.70 |
| | N5♀ | | 205100 | 307.00 | 0.26 | 1.85 | Retired | 7.80 |
| Wild Type | N1♂ | 356 LS | 584100 | 294.17 | 1.27 | 9.16 | Retired | 8.47 |
| Mice | N2♂ | | 700300 | 325.28 | 0.77 | 4.66 | Retired | 9.66 |
| | N3♀ | | 1197000 | 199.28 | 0.55 | 4.07 | Retired | 8.47 |
| | N4♀ | | 1205000 | 144.88 | 0.57 | 4.03 | Retired | 8.85 |
| huFcγR | N1♂ | 356 ALE | 102500 | 303.04 | 0.43 | 3.12 | Retired | 6.62 |
| Transgenic | N2♂ | LS | 308700 | 284.54 | 0.48 | 3.67 | Retired | 7.30 |
| Mice | N3♀ | | 371300 | 153.85 | 0.35 | 2.59 | Retired | 5.75 |
| | N4♀ | | 739900 | 223.05 | 0.31 | 2.31 | Retired | 6.82 |
| | N5♀ | | 1565000 | 265.01 | 0.92 | 6.41 | Retired | 9.51 |
| Wild Type | N1♂ | 356 ALE | 2123000 | 426.22 | 0.97 | 6.31 | Retired | 8.45 |
| Mice | N2♂ | LS | 1419000 | 165.01 | 0.62 | 4.39 | Retired | 7.74 |
| | N3♂ | | 582000 | 246.46 | 0.57 | 4.91 | Retired | 8.62 |
| | N4♀ | | 1032000 | 162.56 | 0.43 | 3.84 | Retired | 9.33 |
| huFcγR | N1♂ | 356 LAGA | 707600 | 328.65 | 0.55 | 3.61 | Retired | 10.30 |
| Transgenic | N2♀ | LS | 706500 | 83.93 | 0.15 | 1.33 | Retired | 7.78 |
| Mice | N3♀ | | 1568000 | 477.06 | 0.50 | 3.35 | Retired | 10.30 |
| | N4♀ | | 1255000 | 132.32 | 0.27 | 2.19 | Retired | 10.00 |
| | N5♀ | | 1132000 | 225.07 | 0.55 | 4.36 | Retired | 9.71 |
| Wild Type | N1♂ | 356 LAGA | 662800 | 231.51 | 0.73 | 4.21 | Retired | 8.31 |
| Mice | N2♂ | LS | 718300 | 382.94 | 0.96 | 5.23 | Retired | 8.15 |
| | N3♀ | | 2117000 | 171.71 | 0.49 | 3.50 | Retired | 11.20 |
| | N4♀ | | 1011000 | 211.51 | 0.53 | 3.83 | Retired | 10.40 |

TABLE 19
Summary of study data: 100 μg dose groups

| | | | | | | | | Antibody Serum |
|---|---|---|---|---|---|---|---|---|
| | | | | qPCR | FACS | FACS | FACS | Concentration |
| Genetic | | Treatment | Total Flux | Parasites/ | % | % | % | (mg/ml) |
| Background | Animal | Dose | p/s | ml | Parasitemia | Parasitemia | Parasitemia | 24 hour after IV |
| Mice | ID | (100 μg) | 44 hours | Day 3 | Day 5 | Day 6 | Day 8 | administration |
| huFcγR | N1♂ | 356 LS | 22790 | 50.78 | 0.17 | 1.32 | Retired | 21.70 |
| Transgenic | N2♂ | | 114900 | 30.25 | 0.39 | 3.13 | Retired | 22.40 |
| Mice | N3♂ | | 70220 | 203.66 | 0.35 | 2.29 | Retired | 34.30 |
| | N4♀ | | 622000 | 141.67 | 0.33 | 2.47 | Retired | 36.80 |
| | N5♀ | | 819400 | 133.70 | 0.22 | 1.87 | Retired | 39.50 |
| Wild Type | N1♀ | 356 LS | 533400 | 116.10 | 0.19 | 1.58 | Retired | 35.30 |
| Mice | N2♀ | | 386000 | 46.85 | 0.09 | 0.70 | 6.74 | 44.70 |
| | N3♀ | | 571500 | 169.20 | 0.31 | 2.03 | Retired | 42.90 |
| | N4♂ | | 2016 | 47.16 | 0.11 | 0.96 | 9.23 | 35.30 |
| huFcγR | N1♂ | 356 ALE | 18950 | 76.86 | 0.08 | 0.51 | 6.65 | 33.10 |
| Transgenic | N2♂ | LS | 960500 | 426.43 | 1.06 | 5.84 | Retired | 6.32 |
| Mice | N3♂ | | 31900 | 44.45 | 0.10 | 0.78 | 6.76 | 26.20 |
| | N4♀ | | 148100 | 20.92 | 0.07 | 0.39 | 10.22 | 40.00 |
| | N5♀ | | 62520 | 137.60 | 0.21 | 1.56 | Retired | 35.10 |
| Wild Type | N1♂ | 356 ALE | 81170 | 69.48 | 0.10 | 0.72 | 6.89 | 31.80 |
| Mice | N2♂ | LS | 86130 | 91.81 | 0.14 | 1.02 | Retired | 26.10 |
| | N3♀ | | 133300 | 101.11 | 0.10 | 0.60 | 11.29 | 44.70 |
| | N4♀ | | 489200 | 26.59 | 0.05 | 0.61 | 6.47 | 39.40 |
| huFcγR | N1♂ | 356 | 41380 | 130.62 | 0.32 | 2.29 | Retired | 23.00 |
| Transgenic | N2♂ | LAGA LS | 14620 | 18.98 | 0.07 | 0.46 | 8.65 | 32.20 |
| Mice | N3♂ | | 275100 | 88.95 | 0.12 | 0.89 | 5.10 | 37.40 |
| | N4♀ | | 189600 | 39.10 | 0.10 | 0.69 | 4.26 | 36.00 |
| | N5♀ | | 716600 | 51.54 | 0.16 | 1.21 | Retired | 43.30 |
| Wild Type | N1♂ | 356 | 91610 | 67.09 | 0.19 | 1.71 | Retired | 32.80 |
| Mice | N2♀ | LAGA LS | 186200 | 45.85 | 0.08 | 0.47 | 7.20 | 46.70 |
| | N3♀ | | 167000 | 11.93 | 0.05 | 0.16 | 8.56 | 47.70 |
| | N4♀ | | 321100 | 33.26 | 0.07 | 0.49 | 5.72 | 39.90 |

Despite showing that NK cells isolated from human transgenic mice respond strongly to Fc enhanced 356-ALE-LS there is no clear increase in protection after sporozoite challenge. The reason for this is unclear but may be due to a range of factors including differences between the mouse challenge model and human, for example the presence and activity of specific effector cell subsets that are thought to be involved in human malaria disease protection (Ty et al, 2023, Sci Transl Med, 15(680):eadd901) which have not been evaluated in the mouse, the difference in time interval between infectious sporozoite challenge and the end of liver stage of disease in mouse compared to human (approximately 2 days compared to 7 days respectively), and, the protocol used in these i.v challenge studies which does not reflect the natural mosquito bite infection route.

### Example 8—In Vivo Evaluation of Antibodies in Mice Engrafted with Human Hepatocytes and Infected with Plasmodium falciparum

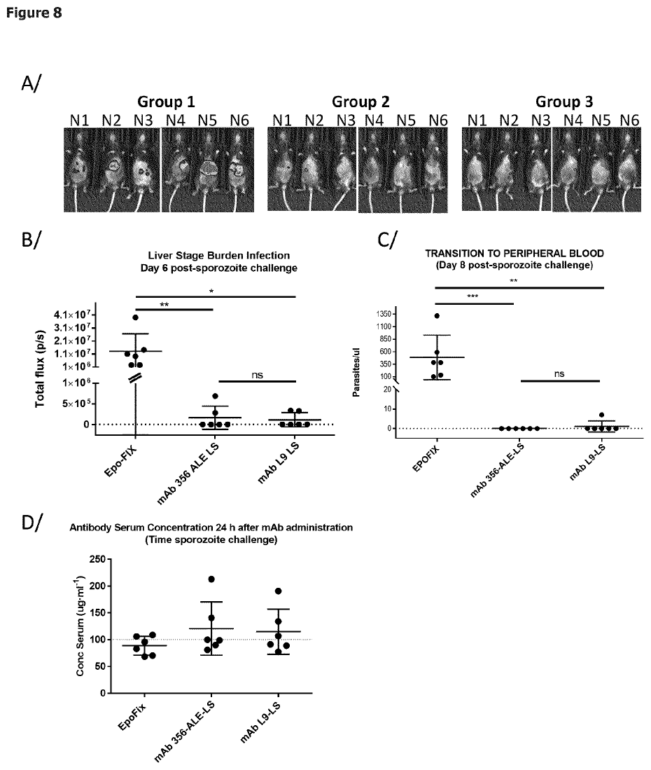

In order to evaluate the in vivo effectiveness of 356-ALE-LS against human malaria P. falciparum, the human liver-chimeric FRG huHep mouse model was used (Minkah et al, 2018, Front Immunol, 19(9):807). These mice are engrafted with primary human hepatocytes, are susceptible to P. falciparum sporozoite infection and support complete liver-stage development. Mice (n=6 per group) were challenged by mosquito bite using 40 infected mosquitoes and 356-ALE-LS (Group 2) administered at a single 300 μg dose per mouse. Antibody L9-LS (Group 3) that has been shown to be highly effective in the FRG model and has recently completed a human malaria challenge study in human was also included in the study as a positive bench-mark control. EpoFix isotype antibody was used as negative control (Group 1). All six mice treated with 356-ALE-LS had undetectable parasitemia in peripheral blood at day 8 post sporozoite challenge which compares to 5 of 6 fully protected mice in the L9-LS group (FIG. 8C). No differences were observed in antibody serum concentrations throughout the study (FIG. 8D). This data shows that 356-ALE-LS is comparable to L9-LS although since the background strain of mice used are WT C57BL/6 the added benefit of improved effector function will not be evident.

The binding affinities of 356-ALE-LS to recombinant CSP was measured by SPR. Data is shown in Table 20. Two different batches of 356 ALE-LS show binding to target recombinant CSP with similar nM affinity.

TABLE 20
Binding affinities of antibodies to recombinant CSP by SPR

| | Geometric | Standard | |
|---|---|---|---|
| mAb | Mean KD (nM) | deviation | n |
| mAb A | 0.10 | 0.012 | 3 |
| L9 | 0.04 | 0.005 | 2 |
| 356-ALE-LS (batch 1) | 1.58 | | 1 |
| 356-ALE-LS (batch 2) | 1.10 | 0.376 | 3 |

The binding affinity of 356 ALE-LS to human Fcγ receptors was measured by SPR. Binding affinities were also determined for 356 LS and the Fc silenced variant 356 LAGA-LS. A human IgG1 antibody in both WT and Fc silenced (LAGA) formats were included as negative control. The data is shown in table 21. mAb 356-ALE-LS shows higher human FcγRIIa and FcγRIIIa binding affinity with similar low hFcγRIIb binding compared to the parental 356-LS mAb.

TABLE 21
Binding affinities of antibodies to human FcγR by SPR
KD (nM)

| mAb | hFcγRI | hFcγRIIaH | hFcγRIIaR | hFcγRIIb | hFcγRIIIaV | hFcγRIIIaF |
|---|---|---|---|---|---|---|
| 356-ALE- | 56 | 218 | 675 | 3893 | 73 | 89 |
| LS | | | | | | |
| (batch 1) | | | | | | |
| 356-ALE- | 157 | 268 | 766 | WB | 144 | 246 |
| LS | | | | | | |
| (batch 2) | | | | | | |
| 356-LS | 169 | 777 | 1414 | 6765 | 291 | 1433 |
| 356- | WB | WB | WB | WB | WB | WB |
| LAGA-LS | | | | | | |
| hIgG1 | WB | WB | WB | WB | WB | WB |
| WT | | | | | | |
| control- | | | | | | |
| LAGA | | | | | | |
| hIgG1 | 94 | 1092 | 1665 | 3003 | 495 | 1166 |
| WT | | | | | | |
| control | | | | | | |

WB = weak binding

The binding affinity of 356 ALE-LS to human FcRn was measured by SPR. Binding affinities were also measured for mAb-LS and two controls, a human IgG1 WT antibody and antibody L9 without LS. The data is shown in Table 22 with the 356-ALE-LS and mAb A LS both showing the anticipated lower binding affinity at pH 6.0.

TABLE 22
Human FcRn binding by SPR

| mAb | KD (nM) at pH 6.0 | KD (nM) at pH 7.0 |
|---|---|---|
| hIgG1 WT control | 102 | NB |
| 356-ALE-LS | 52 | WB |
| mAb A-LS | 62 | WB |
| L9 | 174 | NB |

WB = weak binding
NB = no binding

SEQUENCE LISTING

SEQ ID NO: 1: CDRH1 of 356
NFGMH
SEQ ID NO: 2: CDRH2 356
VIWHDGSNKFYADSVEG
SEQ ID NO: 3: CDRH3
DSLFYDHDNSGYYGY
SEQ ID NO: 4: CDRL1
RASRSVTSKLA
SEQ ID NO: 5: CDRL2
GASTRAT
SEQ ID NO: 6: CDRL3
QQYNNGFT
SEQ ID NO: 7: Variable Heavy Chain
QVQLVESGGGVVQPGRSLRLSCAASGFTFRNFGMHWVRQTPGKGLEWVAVIWHDGSNKFYADSVEGRFTISRDNSK
NMIYLQMNSLRVEDTAIYYCARDSLFYDHDNSGYYGYWGQGTLVTVSS
SEQ ID NO: 8: Variable Light Chain
QIVMTQSPATVSVSPGERATLSCRASRSVTSKLAWYQQKPGQAPRLLIYGASTRATGIPARFSGSGSGTEFTLTISSLQ
SEDFAVYFCQQYNNGFTFGPGTKVDFK
SEQ ID NO: 9: Heavy Chain
QVQLVESGGGVVQPGRSLRLSCAASGFTFRNFGMHWVRQTPGKGLEWVAVIWHDGSNKFYADSVEGRFTISRDNSK
NMIYLQMNSLRVEDTAIYYCARDSLFYDHDNSGYYGYWGQGTLVTVSSASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSCDKTHTC
PPCPAPELLAGPSVFLFPPKPKDTLMISRTPEVTCVVVDVSHEDPEVKFNWYVDGVEVHNAKTKPREEQYNSTYRVVSV
LTVLHQDWLNGKEYKCKVSNKALPLPEEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVLHEALHSHYTQKSLSLSPGK
SEQ ID NO: 10: Light Chain
QIVMTQSPATVSVSPGERATLSCRASRSVTSKLAWYQQKPGQAPRLLIYGASTRATGIPARFSGSGSGTEFTLTISSLQ
SEDFAVYFCQQYNNGFTFGPGTKVDFKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
SEQ ID NO: 11 CDRH1
TYGMH
SEQ ID NO: 12 CDRH2
IIWHDGSKEFYADSVKG
SEQ ID NO: 13 CDRH3
DDFDSSGHSYFHY
SEQ ID NO: 14 CDRL1
RSSQSLVHSDGNTYVH
SEQ ID NO: 15 CDRL2
KVSNRDS
SEQ ID NO: 16 CDRL3
MQGTQWWT
SEQ ID NO: 17 variable heavy chain
QVQLVESGGGVVQPGRSLRLSCAASGFSFSTYGMHWIRQVPGKGLEWVAIIWHDGSKEFYADSVKGRFTISRDNSKK
KLYLQMNSLRAEDTAIYYCVKDDFDSSGHSYFHYWGQGTLVTVSS
SEQ ID NO: 18 variable light chain
GVVMTQSPLSLPVTLGQPASISCRSSQSLVHSDGNTYVHWFQQRPGQSPRRLIYKVSNRDSGVPDRFSGSGSGTDFT
LKISRVEAEDVGVYYCMQGTQWWTFGQGTKVEIK
SEQ ID NO: 19 heavy chain
QVQLVESGGGVVQPGRSLRLSCAASGFSFSTYGMHWIRQVPGKGLEWVAIIWHDGSKEFYADSVKGRFTISRDNSKK
KLYLQMNSLRAEDTAIYYCVKDDFDSSGHSYFHYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKSCDKTHTCPPC
PAPELLAGPSVFLFPPKPKDTLMISRTPEVTCVVVDVSHEDPEVKFNWYVDGVEVHNAKTKPREEQYNSTYRVVSVLTV
LHQDWLNGKEYKCKVSNKALPLPEEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVLHEALHSHYTQKSLSLSPGK
SEQ ID NO: 20 light chain
GVVMTQSPLSLPVTLGQPASISCRSSQSLVHSDGNTYVHWFQQRPGQSPRRLIYKVSNRDSGVPDRFSGSGSGTDFT
LKISRVEAEDVGVYYCMQGTQWWTFGQGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSFNRGEC
SEQ ID NO: 21 variable heavy chain nucleotide sequence (356)
CAGGTGCAGCTGGTGGAGAGCGGCGGCGGCGTGGTGCAGCCAGGCAGATCTCTGAGGCTGAGCTGTGCCGCCAG
CGGCTTTACCTTCAGGAACTTCGGCATGCACTGGGTGAGGCAGACCCCCGGAAAAGGCCTGGAATGGGTGGCCGT
GATTTGGCACGACGGCAGCAACAAATTCTACGCCGACAGCGTGGAGGGCAGGTTCACCATCAGCAGGGACAACAG
CAAGAACATGATCTACCTGCAGATGAACAGCCTGAGGGTGGAGGACACCGCCATCTACTACTGCGCCAGGGACTCT
CTGTTCTACGACCACGACAACTCTGGCTACTACGGCTACTGGGGACAGGGCACTCTGGTGACCGTGAGCAGC
SEQ ID NO: 22 variable light chain nucleotide sequence (356)
CAGATCGTGATGACCCAGAGCCCCGCCACCGTGAGCGTGAGCCCAGGAGAAAGGGCCACTCTGAGCTGCAGGGCA
AGCAGGTCTGTGACCAGCAAGCTGGCATGGTACCAGCAGAAACCCGGCCAGGCTCCCAGGCTGCTGATCTATGGA
GCCAGCACCAGGGCTACCGGCATTCCTGCCAGGTTTAGCGGAAGCGGCAGCGGCACCGAGTTCACCCTGACCATC
TCTAGCCTGCAGAGCGAGGACTTCGCCGTGTACTTCTGCCAGCAGTACAACAACGGCTTCACCTTCGGCCCCGGCA
CCAAGGTGGACTTCAAG
SEQ ID NO: 23 heavy chain nucleotide sequence
CAGGTGCAGCTGGTGGAGAGCGGCGGCGGCGTGGTGCAGCCAGGCAGATCTCTGAGGCTGAGCTGTGCCGCCAG
CGGCTTTACCTTCAGGAACTTCGGCATGCACTGGGTGAGGCAGACCCCCGGAAAAGGCCTGGAATGGGTGGCCGT
GATTTGGCACGACGGCAGCAACAAATTCTACGCCGACAGCGTGGAGGGCAGGTTCACCATCAGCAGGGACAACAG
CAAGAACATGATCTACCTGCAGATGAACAGCCTGAGGGTGGAGGACACCGCCATCTACTACTGCGCCAGGGACTCT
CTGTTCTACGACCACGACAACTCTGGCTACTACGGCTACTGGGGACAGGGCACTCTGGTGACCGTGAGCAGCGCC
AGCACCAAGGGCCCCAGCGTGTTCCCCCTGGCCCCCAGCAGCAAGAGCACCAGCGGCGGCACAGCCGCCCTGGGC
TGCCTGGTGAAGGACTACTTCCCCGAGCCCGTGACCGTGTCCTGGAACAGCGGAGCCCTGACCAGCGGCGTGCAC
ACCTTCCCCGCCGTGCTGCAGAGCAGCGGCCTGTACAGCCTGAGCAGCGTGGTGACCGTGCCCAGCAGCAGCCTG
GGCACCCAGACCTACATCTGTAACGTGAACCACAAGCCCAGCAACACCAAGGTGGACAAGAAGGTGGAGCCCAAGA
GCTGTGACAAGACCCACACCTGCCCCCCCTGCCCTGCCCCCGAGCTGCTGGCCGGCCCCAGCGTGTTCCTGTTCCC
CCCCAAGCCTAAGGACACCCTGATGATCAGCAGAACCCCCGAGGTGACCTGTGTGGTGGTGGATGTGAGCCACGA
GGACCCTGAGGTGAAGTTCAACTGGTACGTGGACGGCGTGGAGGTGCACAATGCCAAGACCAAGCCCAGGGAGGA
GCAGTACAACAGCACCTACCGGGTGGTGTCCGTGCTGACCGTGCTGCACCAGGATTGGCTGAACGGCAAGGAGTA
CAAGTGTAAGGTGTCCAACAAGGCCCTGCCTCTGCCTGAAGAGAAAACCATCAGCAAGGCCAAGGGCCAGCCCAG
AGAGCCCCAGGTGTACACCCTGCCCCCTAGCAGAGATGAGCTGACCAAGAACCAGGTGTCCCTGACCTGCCTGGT
GAAGGGCTTCTACCCCAGCGACATCGCCGTGGAGTGGGAGAGCAACGGCCAGCCCGAGAACAACTACAAGACCAC
CCCCCCTGTGCTGGACAGCGATGGCAGCTTCTTCCTGTACAGCAAGCTGACCGTGGACAAGAGCAGATGGCAGCA
GGGCAACGTGTTCAGCTGCTCCGTGCTGCACGAGGCCCTGCACTCCCACTACACCCAGAAAAGCCTGAGCCTGTCC
CCTGGCAAG
SEQ ID NO: 24 variable light chain nucleotide sequence (356)
CAGATCGTGATGACCCAGAGCCCCGCCACCGTGAGCGTGAGCCCAGGAGAAAGGGCCACTCTGAGCTGCAGGGCA
AGCAGGTCTGTGACCAGCAAGCTGGCATGGTACCAGCAGAAACCCGGCCAGGCTCCCAGGCTGCTGATCTATGGA
GCCAGCACCAGGGCTACCGGCATTCCTGCCAGGTTTAGCGGAAGCGGCAGCGGCACCGAGTTCACCCTGACCATC
TCTAGCCTGCAGAGCGAGGACTTCGCCGTGTACTTCTGCCAGCAGTACAACAACGGCTTCACCTTCGGCCCCGGCA
CCAAGGTGGACTTCAAGCGTACGGTGGCCGCCCCCAGCGTGTTCATCTTCCCCCCCAGCGATGAGCAGCTGAAGA
GCGGCACCGCCAGCGTGGTGTGTCTGCTGAACAACTTCTACCCCCGGGAGGCCAAGGTGCAGTGGAAGGTGGACA
ATGCCCTGCAGAGCGGCAACAGCCAGGAGAGCGTGACCGAGCAGGACAGCAAGGACTCCACCTACAGCCTGAGCA
GCACCCTGACCCTGAGCAAGGCCGACTACGAGAAGCACAAGGTGTACGCCTGTGAGGTGACCCACCAGGGCCTGT
CCAGCCCCGTGACCAAGAGCTTCAACCGGGGCGAGTGC
SEQ ID NO: 25 variable heavy chain nucleotide sequence (338)
CAGGTGCAGCTGGTGGAGAGCGGCGGCGGCGTGGTGCAGCCCGGAAGATCTCTGAGACTGAGCTGTGCCGCCAG
CGGCTTCAGCTTCAGCACCTACGGCATGCATTGGATCAGGCAGGTGCCCGGCAAAGGCCTGGAATGGGTGGCCAT
CATCTGGCACGATGGCAGCAAGGAGTTCTACGCCGACAGCGTGAAGGGCAGGTTCACCATCAGCAGGGACAACAG
CAAGAAGAAGCTGTACCTGCAGATGAACAGCCTGAGGGCCGAGGACACCGCCATCTACTACTGCGTCAAGGACGA
CTTCGACAGCAGCGGCCACAGCTACTTTCACTACTGGGGCCAGGGAACCCTGGTGACAGTGAGCAGC
SEQ ID NO: 26 variable light chain nucleotide sequence (338)
GGCGTGGTGATGACCCAGAGCCCCCTGAGCCTGCCCGTGACACTGGGCCAGCCAGCAAGCATTAGCTGCAGGAGC
TCTCAGAGCCTGGTGCACAGCGACGGCAACACCTACGTGCACTGGTTTCAGCAGAGGCCCGGCCAGTCTCCCAGG
AGGCTGATCTACAAGGTGAGCAACAGGGATAGCGGCGTGCCCGATAGGTTTAGCGGCAGCGGCAGCGGCACCGAC
TTTACCCTGAAGATCTCTAGGGTGGAGGCCGAGGACGTGGGCGTGTACTATTGCATGCAGGGCACCCAGTGGTGG
ACCTTCGGCCAGGGAACCAAGGTGGAGATCAAG
SEQ ID NO: 27 heavy chain nucleotide sequence (338 ALE LS)
CAGGTGCAGCTGGTGGAGAGCGGCGGCGGCGTGGTGCAGCCCGGAAGATCTCTGAGACTGAGCTGTGCCGCCAG
CGGCTTCAGCTTCAGCACCTACGGCATGCATTGGATCAGGCAGGTGCCCGGCAAAGGCCTGGAATGGGTGGCCAT
CATCTGGCACGATGGCAGCAAGGAGTTCTACGCCGACAGCGTGAAGGGCAGGTTCACCATCAGCAGGGACAACAG
CAAGAAGAAGCTGTACCTGCAGATGAACAGCCTGAGGGCCGAGGACACCGCCATCTACTACTGCGTCAAGGACGA
CTTCGACAGCAGCGGCCACAGCTACTTTCACTACTGGGGCCAGGGAACCCTGGTGACAGTGAGCAGCGCCAGCAC
CAAGGGCCCCAGCGTGTTCCCCCTGGCCCCCAGCAGCAAGAGCACCAGCGGCGGCACAGCCGCCCTGGGCTGCCT
GGTGAAGGACTACTTCCCCGAGCCCGTGACCGTGTCCTGGAACAGCGGAGCCCTGACCAGCGGCGTGCACACCTT
CCCCGCCGTGCTGCAGAGCAGCGGCCTGTACAGCCTGAGCAGCGTGGTGACCGTGCCCAGCAGCAGCCTGGGCAC
CCAGACCTACATCTGTAACGTGAACCACAAGCCCAGCAACACCAAGGTGGACAAGAAGGTGGAGCCCAAGAGCTGT
GACAAGACCCACACCTGCCCCCCCTGCCCTGCCCCCGAGCTGCTGGCCGGCCCCAGCGTGTTCCTGTTCCCCCCCA
AGCCTAAGGACACCCTGATGATCAGCAGAACCCCCGAGGTGACCTGTGTGGTGGTGGATGTGAGCCACGAGGACC
CTGAGGTGAAGTTCAACTGGTACGTGGACGGCGTGGAGGTGCACAATGCCAAGACCAAGCCCAGGGAGGAGCAGT
ACAACAGCACCTACCGGGTGGTGTCCGTGCTGACCGTGCTGCACCAGGATTGGCTGAACGGCAAGGAGTACAAGT
GTAAGGTGTCCAACAAGGCCCTGCCTCTGCCTGAAGAGAAAACCATCAGCAAGGCCAAGGGCCAGCCCAGAGAGC
CCCAGGTGTACACCCTGCCCCCTAGCAGAGATGAGCTGACCAAGAACCAGGTGTCCCTGACCTGCCTGGTGAAGG
GCTTCTACCCCAGCGACATCGCCGTGGAGTGGGAGAGCAACGGCCAGCCCGAGAACAACTACAAGACCACCCCCCC
TGTGCTGGACAGCGATGGCAGCTTCTTCCTGTACAGCAAGCTGACCGTGGACAAGAGCAGATGGCAGCAGGGCAA
CGTGTTCAGCTGCTCCGTGCTGCACGAGGCCCTGCACTCCCACTACACCCAGAAAAGCCTGAGCCTGTCCCCTGGC
AAG
SEQ ID NO: 28 light chain nucleotide sequence (338 ALE LS)
GGCGTGGTGATGACCCAGAGCCCCCTGAGCCTGCCCGTGACACTGGGCCAGCCAGCAAGCATTAGCTGCAGGAGC
TCTCAGAGCCTGGTGCACAGCGACGGCAACACCTACGTGCACTGGTTTCAGCAGAGGCCCGGCCAGTCTCCCAGG
AGGCTGATCTACAAGGTGAGCAACAGGGATAGCGGCGTGCCCGATAGGTTTAGCGGCAGCGGCAGCGGCACCGAC
TTTACCCTGAAGATCTCTAGGGTGGAGGCCGAGGACGTGGGCGTGTACTATTGCATGCAGGGCACCCAGTGGTGG
ACCTTCGGCCAGGGAACCAAGGTGGAGATCAAGCGTACGGTGGCCGCCCCCAGCGTGTTCATCTTCCCCCCCAGC
GATGAGCAGCTGAAGAGCGGCACCGCCAGCGTGGTGTGTCTGCTGAACAACTTCTACCCCCGGGAGGCCAAGGTG
CAGTGGAAGGTGGACAATGCCCTGCAGAGCGGCAACAGCCAGGAGAGCGTGACCGAGCAGGACAGCAAGGACTCC
ACCTACAGCCTGAGCAGCACCCTGACCCTGAGCAAGGCCGACTACGAGAAGCACAAGGTGTACGCCTGTGAGGTG
ACCCACCAGGGCCTGTCCAGCCCCGTGACCAAGAGCTTCAACCGGGGCGAGTGC
SEQ ID NO: 29 356 DEA FV LS heavy chain
QVQLVESGGGVVQPGRSLRLSCAASGFTFRNFGMHWVRQTPGKGLEWVAVIWHDGSNKFYADSVEGRFTISRDNSK
NMIYLQMNSLRVEDTAIYYCARDSLFYDHDNSGYYGYWGQGTLVTVSSASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSCDKTHTC
PPCPAPELLAGPDVFLFPPKPKDVLMISRTPEVTCVVVDVSHEDPEVKFNWYVDGVEVHNFKTKPREEQYNSTYRVVSV
LTVLHQDWLNGKEYKCKVSNKALPAPEEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVLHEALHSHYTQKSLSLSPGK
SEQ ID NO: 30 356 DEA FV LS light chain
QIVMTQSPATVSVSPGERATLSCRASRSVTSKLAWYQQKPGQAPRLLIYGASTRATGIPARFSGSGSGTEFTLTISSLQ
SEDFAVYFCQQYNNGFTFGPGTKVDFKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
SEQ ID NO: 31 356 heavy chain nucleotide sequence
CAGGTGCAGCTGGTGGAGAGCGGCGGCGGCGTGGTGCAGCCAGGCAGATCTCTGAGGCTGAGCTGTGCCGCCAG
CGGCTTTACCTTCAGGAACTTCGGCATGCACTGGGTGAGGCAGACCCCCGGAAAAGGCCTGGAATGGGTGGCCGT
GATTTGGCACGACGGCAGCAACAAATTCTACGCCGACAGCGTGGAGGGCAGGTTCACCATCAGCAGGGACAACAG
CAAGAACATGATCTACCTGCAGATGAACAGCCTGAGGGTGGAGGACACCGCCATCTACTACTGCGCCAGGGACTCT
CTGTTCTACGACCACGACAACTCTGGCTACTACGGCTACTGGGGACAGGGCACTCTGGTGACCGTGAGCAGCGCC
AGCACCAAGGGCCCCAGCGTGTTCCCCCTGGCCCCCAGCAGCAAGAGCACCAGCGGCGGCACAGCCGCCCTGGGC
TGCCTGGTGAAGGACTACTTCCCCGAGCCCGTGACCGTGTCCTGGAACAGCGGAGCCCTGACCAGCGGCGTGCAC
ACCTTCCCCGCCGTGCTGCAGAGCAGCGGCCTGTACAGCCTGAGCAGCGTGGTGACCGTGCCCAGCAGCAGCCTG
GGCACCCAGACCTACATCTGTAACGTGAACCACAAGCCCAGCAACACCAAGGTGGACAAGAAGGTGGAGCCCAAGA
GCTGTGACAAGACCCACACCTGCCCCCCCTGCCCTGCCCCCGAGCTGCTGGCCGGCCCCGACGTGTTCCTGTTCCC
CCCCAAGCCTAAGGACGTGCTGATGATCAGCAGAACCCCCGAGGTGACCTGTGTGGTGGTGGATGTGAGCCACGA
GGACCCTGAGGTGAAGTTCAACTGGTACGTGGACGGCGTGGAGGTGCACAATTTCAAGACCAAGCCCAGGGAGGA
GCAGTACAACAGCACCTACCGGGTGGTGTCCGTGCTGACCGTGCTGCACCAGGATTGGCTGAACGGCAAGGAGTA
CAAGTGTAAGGTGTCCAACAAGGCCCTGCCTGCCCCTGAAGAGAAAACCATCAGCAAGGCCAAGGGCCAGCCCAG
AGAGCCCCAGGTGTACACCCTGCCCCCTAGCAGAGATGAGCTGACCAAGAACCAGGTGTCCCTGACCTGCCTGGT
GAAGGGCTTCTACCCCAGCGACATCGCCGTGGAGTGGGAGAGCAACGGCCAGCCCGAGAACAACTACAAGACCAC
CCCCCCTGTGCTGGACAGCGATGGCAGCTTCTTCCTGTACAGCAAGCTGACCGTGGACAAGAGCAGATGGCAGCA
GGGCAACGTGTTCAGCTGCTCCGTGCTGCACGAGGCCCTGCACTCCCACTACACCCAGAAAAGCCTGAGCCTGTCC
CCTGGCAAG
SEQ ID NO: 32 356 DEA FV LS heavy chain nucleotide sequence
CAGATCGTGATGACCCAGAGCCCCGCCACCGTGAGCGTGAGCCCAGGAGAAAGGGCCACTCTGAGCTGCAGGGCA
AGCAGGTCTGTGACCAGCAAGCTGGCATGGTACCAGCAGAAACCCGGCCAGGCTCCCAGGCTGCTGATCTATGGA
GCCAGCACCAGGGCTACCGGCATTCCTGCCAGGTTTAGCGGAAGCGGCAGCGGCACCGAGTTCACCCTGACCATC
TCTAGCCTGCAGAGCGAGGACTTCGCCGTGTACTTCTGCCAGCAGTACAACAACGGCTTCACCTTCGGCCCCGGCA
CCAAGGTGGACTTCAAGCGTACGGTGGCCGCCCCCAGCGTGTTCATCTTCCCCCCCAGCGATGAGCAGCTGAAGA
GCGGCACCGCCAGCGTGGTGTGTCTGCTGAACAACTTCTACCCCCGGGAGGCCAAGGTGCAGTGGAAGGTGGACA
ATGCCCTGCAGAGCGGCAACAGCCAGGAGAGCGTGACCGAGCAGGACAGCAAGGACTCCACCTACAGCCTGAGCA
GCACCCTGACCCTGAGCAAGGCCGACTACGAGAAGCACAAGGTGTACGCCTGTGAGGTGACCCACCAGGGCCTGT
CCAGCCCCGTGACCAAGAGCTTCAACCGGGGCGAGTGC

1. A Plasmodium falciparum: circumsporozoite protein (CSP) antigen binding protein comprising:
a Heavy Chain (HC) sequence of SEQ ID NO: 9; and
a Light Chain (LC) sequence of SEQ ID NO: 10.

2. A CSP antigen binding protein according to claim 1 wherein the antigen binding protein binds with an affinity of at least 2.5 nM when measured by Surface Plasmon Resonance (SPR).

3. A CSP binding protein according to claim 1 wherein the antigen binding protein remains in circulation for a period of at least 3 months after administration.

4. A nucleic acid sequence encoding the heavy chain of SEQ ID NO:9; and/or a nucleic acid sequence encoding the light chain of SEQ ID NO: 10.

5. An expression vector or vectors comprising a nucleic acid sequence encoding the heavy chain of SEQ ID NO:9; and/or a nucleic acid sequence encoding the light chain of SEQ ID NO: 10.

6. A recombinant host cell comprising the nucleic acid sequence(s) as defined in claim 4.

7. A method for the production of a CSP antigen binding protein according to claim 1, which method comprises culturing a recombinant host cell comprising a nucleic acid sequence encoding the heavy chain of SEQ ID NO:9; and/or a nucleic acid sequence encoding the light chain of SEQ ID NO: 10 under conditions suitable for expression of said nucleic acid sequence(s) or vector(s), whereby a polypeptide comprising the CSP antigen binding protein is produced.

8. A pharmaceutical composition comprising the CSP antigen binding protein as defined in claim 1 and a pharmaceutically acceptable excipient.

9. A method for the prevention of malaria in a subject in need thereof comprising administering to said subject a therapeutically effective amount of the pharmaceutical composition as defined in claim 8.

10. (canceled)

11. A pharmaceutical composition as defined in claim 8 for intravenous, subcutaneous or intramuscular administration.

12. (canceled)

13. A recombinant host cell comprising or the expression vector(s) as defined in claim 5.


SEQUENCE LISTING (32 sequences)

SEQ ID NO: 1 - protein, 5 aa, synthetic construct
NFGMH

SEQ ID NO: 2 - protein, 17 aa, synthetic construct
VIWHDGSNKFYADSVEG

SEQ ID NO: 3 - protein, 15 aa, synthetic construct
DSLFYDHDNSGYYGY

SEQ ID NO: 4 - protein, 11 aa, synthetic construct
RASRSVTSKLA

SEQ ID NO: 5 - protein, 7 aa, synthetic construct
GASTRAT

SEQ ID NO: 6 - protein, 8 aa, synthetic construct
QQYNNGFT

SEQ ID NO: 7 - protein, 124 aa, synthetic construct
QVQLVESGGGVVQPGRSLRLSCAASGFTFRNFGMHWVRQTPGKGLEWVAVIWHDGSNKFYADSVEGRFTISRDNSKNMIYLQMNSLRVEDTAIYYCARDSLFYDHDNSGYYGYWGQGTLVTVSS

SEQ ID NO: 8 - protein, 106 aa, synthetic construct
QIVMTQSPATVSVSPGERATLSCRASRSVTSKLAWYQQKPGQAPRLLIYGASTRATGIPARFSGSGSGTEFTLTISSLQSEDFAVYFCQQYNNGFTFGPGTKVDFK

SEQ ID NO: 9 - protein, 454 aa, synthetic construct
QVQLVESGGGVVQPGRSLRLSCAASGFTFRNFGMHWVRQTPGKGLEWVAVIWHDGSNKFYADSVEGRFTISRDNSKNMIYLQMNSLRVEDTAIYYCARDSLFYDHDNSGYYGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 10 - protein, 213 aa, synthetic construct
QIVMTQSPATVSVSPGERATLSCRASRSVTSKLAWYQQKPGQAPRLLIYGASTRATGIPARFSGSGSGTEFTLTISSLQSEDFAVYFCQQYNNGFTFGPGTKVDFKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 5 aa, synthetic construct
TYGMH

SEQ ID NO: 12 - protein, 17 aa, synthetic construct
IIWHDGSKEFYADSVKG

SEQ ID NO: 13 - protein, 13 aa, synthetic construct
DDFDSSGHSYFHY

SEQ ID NO: 14 - protein, 16 aa, synthetic construct
RSSQSLVHSDGNTYVH

SEQ ID NO: 15 - protein, 7 aa, synthetic construct
KVSNRDS

SEQ ID NO: 16 - protein, 8 aa, synthetic construct
MQGTQWWT

SEQ ID NO: 17 - protein, 122 aa, synthetic construct
QVQLVESGGGVVQPGRSLRLSCAASGFSFSTYGMHWIRQVPGKGLEWVAIIWHDGSKEFYADSVKGRFTISRDNSKKKLYLQMNSLRAEDTAIYYCVKDDFDSSGHSYFHYWGQGTLVTVSS

SEQ ID NO: 18 - protein, 111 aa, synthetic construct
GVVMTQSPLSLPVTLGQPASISCRSSQSLVHSDGNTYVHWFQQRPGQSPRRLIYKVSNRDSGVPDRFSGSGSGTDFTLKISRVEAEDVGVYYCMQGTQWWTFGQGTKVEIK

SEQ ID NO: 19 - protein, 452 aa, synthetic construct
QVQLVESGGGVVQPGRSLRLSCAASGFSFSTYGMHWIRQVPGKGLEWVAIIWHDGSKEFYADSVKGRFTISRDNSKKKLYLQMNSLRAEDTAIYYCVKDDFDSSGHSYFHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 20 - protein, 218 aa, synthetic construct
GVVMTQSPLSLPVTLGQPASISCRSSQSLVHSDGNTYVHWFQQRPGQSPRRLIYKVSNRDSGVPDRFSGSGSGTDFTLKISRVEAEDVGVYYCMQGTQWWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 372 bp, synthetic construct
  source 1..372: mol_type = other DNA
caggtgcagctggtggagagcggcggcggcgtggtgcagccaggcagatctctgaggctgagctgtgccgccagcggctttaccttcaggaacttcggcatgcactgggtgaggcagacccccggaaaaggcctggaatgggtggccgtgatttggcacgacggcagcaacaaattctacgccgacagcgtggagggcaggttcaccatcagcagggacaacagcaagaacatgatctacctgcagatgaacagcctgagggtggaggacaccgccatctactactgcgccagggactctctgttctacgaccacgacaactctggctactacggctactggggacagggcactctggtgaccgtgagcagc

SEQ ID NO: 22 - DNA, 318 bp, synthetic construct
  source 1..318: mol_type = other DNA
cagatcgtgatgacccagagccccgccaccgtgagcgtgagcccaggagaaagggccactctgagctgcagggcaagcaggtctgtgaccagcaagctggcatggtaccagcagaaacccggccaggctcccaggctgctgatctatggagccagcaccagggctaccggcattcctgccaggtttagcggaagcggcagcggcaccgagttcaccctgaccatctctagcctgcagagcgaggacttcgccgtgtacttctgccagcagtacaacaacggcttcaccttcggccccggcaccaaggtggacttcaag

SEQ ID NO: 23 - DNA, 1362 bp, synthetic construct
  source 1..1362: mol_type = other DNA
caggtgcagctggtggagagcggcggcggcgtggtgcagccaggcagatctctgaggctgagctgtgccgccagcggctttaccttcaggaacttcggcatgcactgggtgaggcagacccccggaaaaggcctggaatgggtggccgtgatttggcacgacggcagcaacaaattctacgccgacagcgtggagggcaggttcaccatcagcagggacaacagcaagaacatgatctacctgcagatgaacagcctgagggtggaggacaccgccatctactactgcgccagggactctctgttctacgaccacgacaactctggctactacggctactggggacagggcactctggtgaccgtgagcagcgccagcaccaagggccccagcgtgttccccctggcccccagcagcaagagcaccagcggcggcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctggccggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctctgcctgaagagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgctgcacgaggccctgcactcccactacacccagaaaagcctgagcctgtcccctggcaag

SEQ ID NO: 24 - DNA, 639 bp, synthetic construct
  source 1..639: mol_type = other DNA
cagatcgtgatgacccagagccccgccaccgtgagcgtgagcccaggagaaagggccactctgagctgcagggcaagcaggtctgtgaccagcaagctggcatggtaccagcagaaacccggccaggctcccaggctgctgatctatggagccagcaccagggctaccggcattcctgccaggtttagcggaagcggcagcggcaccgagttcaccctgaccatctctagcctgcagagcgaggacttcgccgtgtacttctgccagcagtacaacaacggcttcaccttcggccccggcaccaaggtggacttcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 25 - DNA, 366 bp, synthetic construct
  source 1..366: mol_type = other DNA
caggtgcagctggtggagagcggcggcggcgtggtgcagcccggaagatctctgagactgagctgtgccgccagcggcttcagcttcagcacctacggcatgcattggatcaggcaggtgcccggcaaaggcctggaatgggtggccatcatctggcacgatggcagcaaggagttctacgccgacagcgtgaagggcaggttcaccatcagcagggacaacagcaagaagaagctgtacctgcagatgaacagcctgagggccgaggacaccgccatctactactgcgtcaaggacgacttcgacagcagcggccacagctactttcactactggggccagggaaccctggtgacagtgagcagc

SEQ ID NO: 26 - DNA, 333 bp, synthetic construct
  source 1..333: mol_type = other DNA
ggcgtggtgatgacccagagccccctgagcctgcccgtgacactgggccagccagcaagcattagctgcaggagctctcagagcctggtgcacagcgacggcaacacctacgtgcactggtttcagcagaggcccggccagtctcccaggaggctgatctacaaggtgagcaacagggatagcggcgtgcccgataggtttagcggcagcggcagcggcaccgactttaccctgaagatctctagggtggaggccgaggacgtgggcgtgtactattgcatgcagggcacccagtggtggaccttcggccagggaaccaaggtggagatcaag

SEQ ID NO: 27 - DNA, 1356 bp, synthetic construct
  source 1..1356: mol_type = other DNA
caggtgcagctggtggagagcggcggcggcgtggtgcagcccggaagatctctgagactgagctgtgccgccagcggcttcagcttcagcacctacggcatgcattggatcaggcaggtgcccggcaaaggcctggaatgggtggccatcatctggcacgatggcagcaaggagttctacgccgacagcgtgaagggcaggttcaccatcagcagggacaacagcaagaagaagctgtacctgcagatgaacagcctgagggccgaggacaccgccatctactactgcgtcaaggacgacttcgacagcagcggccacagctactttcactactggggccagggaaccctggtgacagtgagcagcgccagcaccaagggccccagcgtgttccccctggcccccagcagcaagagcaccagcggcggcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctggccggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctctgcctgaagagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgctgcacgaggccctgcactcccactacacccagaaaagcctgagcctgtcccctggcaag

SEQ ID NO: 28 - DNA, 654 bp, synthetic construct
  source 1..654: mol_type = other DNA
ggcgtggtgatgacccagagccccctgagcctgcccgtgacactgggccagccagcaagcattagctgcaggagctctcagagcctggtgcacagcgacggcaacacctacgtgcactggtttcagcagaggcccggccagtctcccaggaggctgatctacaaggtgagcaacagggatagcggcgtgcccgataggtttagcggcagcggcagcggcaccgactttaccctgaagatctctagggtggaggccgaggacgtgggcgtgtactattgcatgcagggcacccagtggtggaccttcggccagggaaccaaggtggagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 29 - protein, 454 aa, synthetic construct
QVQLVESGGGVVQPGRSLRLSCAASGFTFRNFGMHWVRQTPGKGLEWVAVIWHDGSNKFYADSVEGRFTISRDNSKNMIYLQMNSLRVEDTAIYYCARDSLFYDHDNSGYYGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPDVFLFPPKPKDVLMISRTPEVTCVVVDVSHEDPEVKFNWYVDGVEVHNFKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 30 - protein, 213 aa, synthetic construct; identical to SEQ ID NO: 10

SEQ ID NO: 31 - DNA, 1362 bp, synthetic construct
  source 1..1362: mol_type = other DNA
caggtgcagctggtggagagcggcggcggcgtggtgcagccaggcagatctctgaggctgagctgtgccgccagcggctttaccttcaggaacttcggcatgcactgggtgaggcagacccccggaaaaggcctggaatgggtggccgtgatttggcacgacggcagcaacaaattctacgccgacagcgtggagggcaggttcaccatcagcagggacaacagcaagaacatgatctacctgcagatgaacagcctgagggtggaggacaccgccatctactactgcgccagggactctctgttctacgaccacgacaactctggctactacggctactggggacagggcactctggtgaccgtgagcagcgccagcaccaagggccccagcgtgttccccctggcccccagcagcaagagcaccagcggcggcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctggccggccccgacgtgttcctgttcccccccaagcctaaggacgtgctgatgatcagcagaacccccgaggtgacctgtgtggtggtggatgtgagccacgaggaccctgaggtgaagttcaactggtacgtggacggcgtggaggtgcacaatttcaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctgaagagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgctgcacgaggccctgcactcccactacacccagaaaagcctgagcctgtcccctggcaag

SEQ ID NO: 32 - DNA, 639 bp, synthetic construct; identical to SEQ ID NO: 24
  source 1..639: mol_type = other DNA